(19) United States
(12) Patent Application Publication
(10) Pub. No.: US 2011/0160146 A1
(43) Pub. Date: Jun. 30, 2011

(54) Conjungation of Small Molecules to Octaarginine Transporters for Overcoming Multi-Drug Resistance

(75) Inventors: Paul Wender, Menlo Park, CA (US); Elena A. Dibikovskaya, Richmond, CA (US); Stephen H. Thorne, Pittsburgh, PA (US); Christopher H. Contag, Stanford, CA (US)

(73) Assignee: National Institute of Health (NIH), Bethesda, MD (US)

(21) Appl. No.: 12/865,692

(22) PCT Filed: Feb. 5, 2009
(86) PCT No.: PCT/US2009/000754

§ 371 Date: Feb. 16, 2011

Related U.S. Application Data
(60) Provisional application No. 61/007,508, filed on Feb. 7, 2008.

(51) Int. Cl. A61K 47/48; C12N 5/09; C12N 5/095; C07K 2/00; C07K 7/06; C07K 7/08; A61P 35/00

(52) U.S. Cl. 514/19.3; 435/375; 530/300; 530/329; 530/328; 530/327

(57) ABSTRACT

Many cancer therapeutic agents elicit resistance that renders them ineffective and often produces cross resistance to other drugs. One of the most common mechanisms of resistance involves P-glycoprotein (Pgp) mediated drug efflux. Here we provide compositions and methods that restore the efficacy of a therapeutic agent reduced by resistance by conjugation of the same agent to an oligoarginine transporter comprising from about 5 to about 25 guanidino or amidino moieties. We specifically show that the widely used chemotherapeutic agent taxol, ineffective against taxol-resistant human ovarian cancer cell lines, can be incorporated into an octaarginine conjugate that is effective against the same taxol-resistant cell lines. Significantly, the ability of the taxol conjugates to overcome taxol resistance is observed both in cell culture and in animal models of ovarian cancer. The generality and mechanistic basis for this effect were also explored with other Pgp substrate. This approach shows generality for overcoming the multidrug resistance elicited by small molecule cancer chemotherapeutics and could improve the prognosis for many cancer patients and fundamentally alter search strategies for novel therapeutic agents effective against resistant disease.

### GOVERNMENT RIGHTS

This invention was made with Government support under contracts P50 CA114747-01, CA31841, and CA31845 awarded by the National Institutes of Health. The Government has certain rights in this invention.

Multidrug resistance (MDR) is the resistance of tumor cells to the cytostatic or cytotoxic actions of multiple, structurally dissimilar and functionally divergent drugs commonly used in cancer chemotherapy. It arises from increased expression of membrane proteins that mediate unidirectional energy-dependent drug efflux, thereby intercepting and exporting the drug before it reaches its intracellular target. This type of resistance is general, being observed for many cancer types including those putatively of a stem-like cell origin and for a wide range of chemotherapeutic structural classes operating through a variety of targets and pathways. Moreover, resistance induced by one agent often results in cross resistance to multiple agents. In large part, MDR arises from increased expression of membrane proteins that mediate unidirectional energy-dependent drug efflux. P-glycoprotein (Pgp), a prototypical MDR protein, mediates unidirectional ATP-dependent drug efflux, thereby intercepting and exporting the drug before it reaches its intracellular target. A product of the human MDR1 gene, Pgp belongs to the ATP-binding cassette (ABC) superfamily of small molecule and ion transporters. Other members of the ABC superfamily have also been implicated in multidrug resistance, including multidrug resistance-associated protein-1 (MRP1), its homologs MRP2-6 that transport glutathione, glucuronate and sulfate-conjugated drugs, and the breast cancer resistance protein (BCRP).

Tumors arising from cells that highly express Pgp or other MDR related transporters are often intrinsically resistant to chemotherapy. Other tumor cells acquire high MDR transporter expression upon drug treatment via gene induction or DNA amplification. It is generally believed that these transporters mediate MDR by effecting an export of drugs, thus reducing cellular drug levels and efficacy.

The involvement of Pgp and other MDR-associated transporters in cancer treatment has inspired a search for compounds that inhibit MDR transporters. The initial demonstration of verapamil as a Pgp inhibitor was followed by the identification of other chemosensitizers, or MDR reversal agents, such as calcium channel blockers; cyclosporin A; erythromycin; quinine; phenothiazines and indole alkaloids such as fluphenazine and reserpine; steroid such as progesterone and tamoxifen; and detergents such as cremophor EL.

However, clinical trials with first and second generation MDR drugs failed for various reasons, often due to side effects resulting from adverse reactions to the drug itself. Third generation MRD reversal agents were encouraging in early trials, but for some, such as PSC-833, have revealed potentially significant pharmacokinetic interactions with several anticancer drugs and possible inhibition of non-MDR-related transporters.

The difficulties encountered with direct inhibitors of MDR transporters in the clinic, and the emerging complexity of the MDR phenotype has created interest in alternative approaches to MDR therapy. On approach targets physiological mechanisms involved in regulation of MDR proteins, through manipulation of the signaling pathways that regulate its expression. Such approaches include antagonizing the nuclear steroid and xenobiotic receptor (SXR) to counteract the induction of MDR1; or administering agents that inhibit GlcCer synthase.

Alternative methods have look at circumventing MDR mechanisms, e.g. in developing chemotherapeutic drugs that are poor substrates for MDR transporters; and in the development of cancer vaccines that utilize host immune mechanisms to treat disease.

Overcoming the multidrug resistance elicited by small molecule cancer chemotherapeutics would improve the prognosis for many cancer patients and fundamentally alter the way in which tumors are treated. The present invention provides a general approach to modifying small molecule cancer therapeutics to improve administration, delivery, and efficacy and to overcome resistance.

### SUMMARY OF THE INVENTION

Compositions and methods are provided for the treatment of cancer, including the treatment of multidrug resistant cancer, e.g. in cancer cells that have multidrug resistance mediated by ATP-binding cassette (ABC) superfamily of transporters, such as P-glycoprotein. In the methods of the invention, cancer cells are incubated with a chemotherapeutic agent conjugated to a peptide transporter moiety comprising from about 5 to about 25 guanidino or amidino moieties, more usually from about 6 to about 12 guanidino or amidino moieties. In some embodiments the peptide transporter moiety is an D-octaarginine (r8) transporter.

Compositions of interest for treatment of multidrug resistant cancer include chemotherapeutic drugs, particularly drugs susceptible to multidrug resistance, conjugated to a peptide transporter moiety. Many drugs (e.g., etoposide, camptothecin, and doxorubicin) because of their hydrophobic nature are substrates for Pgp efflux pumps. Attachment of a transporter to these agents dramatically changes their physical properties and mode of cell entry, thereby avoiding Pgp based resistance. In some embodiments, the drug is conjugated to the peptide transporter moiety by a cleavable linker, particularly a linker having a cleavable disulfide bond.

In some embodiments, the chemotherapeutic agent has the structure as follows:

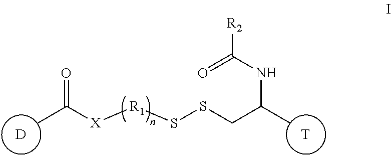

where X is CH₂; C(CH₃)₂; O; NH; or S;
R₁ is CH₂; C(CH₃)₂; C(C₂H₅)₂ or a combination thereof;
R₂ is CH₃, any alkyl chain, e.g. a C₁-C₆ lower alkyl, amino acid or peptide;
n=0, 1, 2, 3, 4, 5;
D is a chemotherapeutic drug; and
T is a guanidinium rich transporter moiety (peptidic or nonpeptidic);
where the linker may be conjugated to a hydroxyl, sulfhydryl, amine, etc. group; and T is usually conjugated to the linker via a disulfide bond, which may be formed by the displacement of the thiopyridyl moiety of the precursor with free thiol of acylated D-cysteine D-octaarginine (AcNHcr8CONH2) to give the transporter-linker conjugate. Drugs of interest include, without limitation, poorly water-soluble therapeutic agents such as taxanes, e.g. paclitaxel, docetaxel, and derivatives and analogs thereof.

In some embodiments of the invention, the cancer cells are tested for multidrug resistance prior to administering the drug conjugate, where a cancer having at least about 10%, at least about 25%, at least about 50% or more cells expressing membrane proteins that mediate unidirectional energy-dependent drug efflux, or actively excluding other substrates is considered positive for multidrug resistance and is treated with the drug conjugates of the invention.

### BRIEF DESCRIPTION OF THE FIGURES

FIG. 1. Mechanisms of action of octaarginine taxol conjugates. (a) Tubulin polymerization assay. The ability of taxol, or octaarginine conjugates at the C2′ (2a) or C7 (3a) position to polymerize free tubulin was determined by increase in turbidity (measured by absorbance at 350 nm). Squares: No drug control; Triangles: taxol; Inverted triangles: octaarginine conjugate to C2′ (2a) (C2′); Diamonds: octaarginine conjugated to C7 (3a) (C7) (b) Cell cycle assay. Cells were treated with 1 mM of the indicated compounds for 15 minutes 24 hours prior to analysis, stained with 7-AAD (7-amino-actomycin D), and analyzed by flow cytometry. Cell doublets were removed and the percentage of single cells in the G2/M interphase (displaying mitotic arrest) were determined. C2′: C2′ octaarginine taxol conjugate (2a); C7: C7 octaarginine taxol conjugate (3a) (*conditions under which octaarginine conjugates produce significantly greater % of cells in G2/M than taxol alone; OVCA429 C2′ p=0.0045, C7 p=0.0051; OVCA429T C7 p=0.0034) (c) Overcoming multidrug resistance through conjugation to octaarginine. OVCA429 (taxol sensitive) and OVCA429T (taxol resistant through complex mechanism including Pgp efflux pump upregulation) cells expressing Renilla luciferase were treated with coelenterazine H (the substrate for Renilla luciferase and a substrate for Pgp), with or without pre-treatement with the Pgp inhibitor cyclosporine A. Subsequent light output, as a measure of drug uptake, was determined by bioluminescence imaging.

FIG. 2. Effect of octaarginine (r8) conjugation on biodistribution of drug. Luciferin, and luciferin conjugated to r8 (7) or k4 (8, tetra-lysine, which has similar water solubility to r8 but limited cellular uptake) were delivered at concentrations equimolar to those used in the studies with taxol (5 mg/kg; FIG. 3) via intraperitoneal injection to transgenic mice ubiquitously expressing Firefly luciferase. (a) Representative image of treated mice at 40 min post injection. (b) Total signal per mouse at times post injection (average of two experiments).

FIG. 3. Survival of tumor-bearing mice treated with taxol or its derivatives. (a) 1×10⁷ UCI-101 tumor cells expressing luciferase were implanted into the peritoneal cavity of athymic nu/nu mice 7 days prior to treatment. Mice were treated with intraperitoneal injections of 5 mg/kg (left panel) or 10 mg/kg (right panel) of taxol or equimolar amounts of its derivatives (octaarginine conjugated to C2′ (2a) or C7 (3a) positions) on days 0, 5 and 10. For the purpose of these experiments TFA⁻ counteranions on both conjugates were exchanged to C. Subsequent tumor burden was followed by bioluminescence imaging (n=8/group). C7 conjugate (3a) produces significantly greater survival than taxol both at 5 mg/kg (p=0.0039) and 10 mg/kg (p=0.047). (b) Mice were implanted with 1×10⁷ cells of OVCA429 (taxol sensitive), or OVCA429T (taxol resistant) cells expressing luciferase and subsequently treated (7 days later) with 5 mg/kg of taxol or equimolar amounts of an octaarginine C2′ conjugated derivative (2a), on days 0, 5 and 10. Tumor burden was followed by bioluminescence imaging. Kaplan-Meier survival curves are shown. (n=8/group). The C7 octaarginine conjugate (3a) produces significantly better survival than taxol in OVCA429T cells (p=0.0002).

### DETAILED DESCRIPTION OF THE EMBODIMENTS

Chemotherapeutic agents are conjugated to peptide transporter moieties for use in the treatment of multidrug resistant cancers. Although the chemotherapeutic agents can be active in the absence of the transporter moiety, the concentrations required for killing of MDR cancer cells may create unacceptable side effects. The conjugated compounds set forth in the present invention provide for an improved therapeutic index, particularly with multidrug resistant cancers. The cancer cells are incubated with the conjugates in vitro or in vivo under physiological conditions. The subject methods also provide a means for therapeutic treatment and investigation of hyperproliferative disorders. Animal models, particularly small mammals, such as murine, lagomorpha, etc., for example where human ovarian cancer cells are injected in immunodeficient mice, are of interest for experimental investigations.

The peptide transporter moieties of the present invention are highly water soluble, charged peptides that are shown herein to enhance the aqueous solubility and cellular uptake of therapeutic molecules and to minimize off-target effects that can limit therapeutic efficacy. The improved activity is demonstrated for multiple P-glycoprotein substrates, showing the generality of the method.

Conjugates of poorly water-soluble therapeutic agents, such as taxol, exhibit greatly improved aqueous solubility, an enhanced therapeutic index, and, significantly, activity against resistance elicited by the drug alone. These conjugates may link the chemotherapeutic drug to the transporter moiety by a disulfide bond, which allows for sustained release of the free drug only after cell entry. Such conjugates are readily administered in aqueous solution, thereby avoiding the prolonged administration and usage of toxic solubilizing reagents (such as Chremophor EL) required for the poorly soluble agent alone. The conjugates have displayed dramatically improved cytotoxic activity against human cancer cell lines and in animal models relative to the free drug alone. Significantly, activity against multidrug resistant cancer cell lines was also observed when these conjugates were evaluated in vitro and in vivo. In addition, the transporter conjugate exhibits altered biodistribution, with selective, increased local uptake sufficient to overcome multidrug resistance. The latter can also help to minimize toxicity associated with systemic delivery of the drug.

The invention also provides for a cleavable linker, which is cleaved only after cellular entry of the conjugate, at a rate controlled by linker design. The linker utilizes a carbonate or ester group in combination with a disulfide cleavable moiety. The length of the linker, and selection of carbonate or ester linkage, provides a means of “tuning” the delivery with respect to release rates. Linkers can be attached to the drug at a suitable position, e.g. at a hydroxyl, amine or sulfhydryl group. In some embodiments of the invention, the drug is a taxane, as shown in Formula II, where the linker is attached to the taxane at the C2′, C7, or C10 hydroxyl position (R₁, R₂, and R₃).

For example, free taxol has the structure shown in Formula II, where R₁═R₂═H. The conjugate may have the structure of Formula II, where R₁ is the linker of Formula III and R₂ is H; or where R₁ is H and R₂ is the linker of Formula III. The C2′ conjugate is of particular interest, as the conjugate is inactive until the disulfide is cleaved. Docetaxel differs from paclitaxel at two positions in its chemical structure. It has a hydroxyl functional group on carbon 10, whereas paclitaxel has an acetate ester, and a tert-butyl substitution exists on the phenylpropionate side chain. Docetaxel conjugates at the C7 or C2′ hydroxy position are also provided.

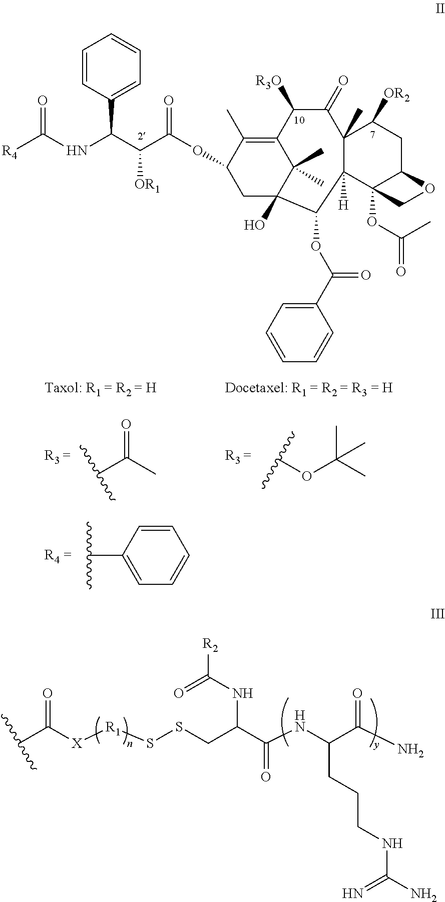

where X is CH₂; C(CH₃)₂; 0; NH; or S;

R₁ is CH₂; C(CH₃)₂; C(C₂H₅)₂ or a combination thereof;

R₂ is CH₃, any alkyl chain, e.g. a C₁-C₆ lower alkyl, amino acid or peptide;

n is 0 to 5, usually from 1-4, more usually 3;

y is 5-12, usually from 6-10, and may be 8.

In addition to paclitaxel and docetaxel, a number of derivatives and analogs are known in the art, for example as described in any one of U.S. Pat. Nos. 7,276,499; 7,256,213; RE39,723; 6,900,342; 6,649,777; 6,649,632; 6,632,795; 6,610,860; 6,596,757; 6,596,737; 6,589,979; 6,541,242; 6,521,660; 6,515,151; 6,500,966; 6,410,756; 6,359,154; 6,350,887; 6,335,362; 6,291,691; 6,278,026; 6,051,724; 6,028,205; etc. Substantial synthetic and biological information is available on syntheses and activities of a variety of taxane and taxoid compounds, as reviewed in Suffness (1995) Taxol: Science and Applications, CRC Press, New York, N.Y., pp. 237-239, particularly in Chapters 12 to 14, as well as in the subsequent paclitaxel literature. Furthermore, a host of cell lines are available for predicting anticancer activities of these compounds against certain cancer types, as described, for example, in Suffness at Chapters 8 and 13.

It will be appreciated that the taxane conjugates of the invention have improved water solubility relative to taxol (˜0.25 μg/mL) and taxotere (6-7 μg/mL). Therefore, large amounts of solubilizing agents such as “CREMOPHOR EL” (polyoxyethylated castor oil), polysorbate 80 (polyoxyethylene sorbitan monooleate, also known as “TWEEN 80”), and ethanol are not required, so that side-effects typically associated with these solubilizing agents, such as anaphylaxis, dyspnea, hypotension, and flushing, can be reduced. Many other chemotherapeutic drugs (e.g., etoposide, camptothecin, and doxorubicin) because of their hydrophobic nature have similar solubility problems. Attachment of a transporter to these agents could dramatically change their physical properties and help to avoid toxic formulations.

In some embodiments the chemotherapeutic agent is a topoisomerase inhibitor, e.g. anthracyclines, including the compounds daunorubicin, adriamycin (doxorubicin) epirubicin, idarubicin, anamycin, MEN 10755, and the like. Another important class of topoisomerase inhibitors included in the invention is a class of cytotoxic quinoline alkaloids. This class includes camptothecin, SN-38, DX-8951f, topotecan, 9-aminocamptothecin, BN 80915, irinotecan, DB 67, BNP 1350, exatecan, lurtotecan, ST 1481, and CKD 602. Other topoisomerase inhibitors include the podophyllotoxin analogues etoposide and teniposide, and the anthracenediones, mitoxantrone and amsacrine, as well as their derivatives.

In another aspect of the invention, the chemotherapeutic agent interferes with microtubule assembly, e.g. the family of vinca alkaloids, etc. Examples of vinca alkaloids include vinblastine, vincristine; vinorelbine (NAVELBINE); vindesine; vindoline; vincamine; etc. Drug resistance to these compounds is due primarily to decreased drug accumulation and results from overexpression of the P-glycoprotein. The methods of the present invention may find use in preventing the selection of drug resistant cells, and in treating resistant tumors.

In this invention, the delivery enhancing transporter connected to a drug could be any of many types of “molecular transporters”. The term itself is connected to function and thus applies to all structural varieties of agents that enable or enhance passage across biological barriers. This classification by “function” (i.e., transporter) is important because the frequently used classification by “structure” (e.g., peptide) is limiting. For example, the term “cell-penetrating peptides” (CPPs), while accurate in part with respect to function, would be technically and obviously limited to “peptides”. However, as discussed herein, systems have been designed based on peptide leads which, while emulating the cell-penetrating function of the leads, are not themselves peptides. In addition to peptides, peptoids, and oligocarbamates, many other types of molecular transporters could be used in the invention. They cover a range of structural classes, including polyamines, polysaccharides, steroids, cationic lipids, guanidinoglycosides, and even nanotubes. The term molecular transporters thus fully covers these structural variants and their similar function. In addition, many of these transporters can be hybridized (e.g., steroid-modified oligoguanidines) to create new transporter types that could be used in the invention.

In some embodiments the delivery enhancing transporter is a classical CPP. Examples include Tat 9-mer (RKKRRQRRR or Tat₄₉₋₅₇), transportan, penetratin, antennapedia and derivatives of thereof.

The delivery-enhancing transporters have sufficient guanidino or amidino moieties to increase delivery of the conjugate into multidrug resistant cells compared to delivery of the free drug in the absence of the delivery-enhancing transporter. In some embodiments, delivery of the conjugate into multidrug resistant cells is increased at least two-fold compared to delivery of the drug in the absence of the delivery-enhancing transporter. In more preferred embodiments, delivery of the conjugate into the multidrug resistant cells is increased at least ten-fold compared to delivery of the drug in the absence of the delivery-enhancing transporter.

In some embodiments, the delivery-enhancing transporter comprises at least about 5, at least about 6, at least about 7, at least about 8, and not more than about 15, not more than about 12, arginine residues or analogs of arginine. The delivery-enhancing transporter can have at least one arginine that is a D-arginine and in some embodiments, all arginines are D-arginine. In some embodiments, at least 70% of the amino acids are arginines or arginine analogs. In some embodiments, the delivery-enhancing transporter comprises at least 5 contiguous arginines or arginine analogs. The delivery-enhancing transporters can comprise non-peptide backbones.

As discussed above, the compound to be delivered can be connected to the delivery-enhancing transporter by a linker. In some embodiments, the linker is a releasable linker which releases the compound, in biologically active form, from the delivery-enhancing transporter after the compound has passed into the cell. In particular, carbamate, ester, thioether, disulfide, and hydrazone linkages are generally easy to form and suitable for most applications. Other linkers such as trimethyl lock (see Wang et. al. J. Org. Chem., 62:1363 (1997) and Chandran et al., J. Am. Chem. Soc., 127:1652 (2005)), quinine methide linker (see Greenwald et. al. J. Med. Chem., 42:3657 (1999) and Greenwald et. al. Bioconjugate Chem., 14:395 (2003)), diketopiperazine linker and derivatives of thereof are also of interest of this invention.

The subject methods are used for prophylactic or therapeutic purposes. The term “treatment” as used herein refers to reducing or alleviating symptoms in a subject, preventing symptoms from worsening or progressing, inhibition or elimination of the causative agent, or prevention of the disorder in a subject who is free therefrom. For example, treatment of a cancer patient may be reduction of tumor size, elimination of malignant cells, prevention of metastasis, or the prevention of relapse in a patient whose tumor has regressed. The treatment of ongoing disease, to stabilize or improve the clinical symptoms of the patient, is of particular interest. Such treatment is desirably performed prior to complete loss of function in the affected tissues.

Multidrug resistant cancer. As used herein, the term “multidrug resistant”, or “MDR” cancer refers to cancer cells that intrinsically or by acquired means are resistant to multiple classes of chemotherapeutic agents. A number of tumors overexpress the MDR-1 gene; including neuroblastoma, rhabdomyosarcoma, myeloma, non-Hodgkin's lymphomas, colon carcinoma, ovarian, breast carcinoma and renal cell cancer. Several tumor types with high MDR-1 expression derive from tissues that have a high expression of the gene, e.g. colonic epithelium. As a non-limiting example, such cells may be resistant to the spectrum of agents including: paclitaxel, doxorubicin, daunorubicin, mitoxantrone, actinomycin D, plicamycin, vincristine, vinblastine, colchicine, etoposide, teniposide, camptothecin and derivatives of thereof. By resistant, it is intended that the IC₅₀ (the half maximal (50%) inhibitory concentration) of the drug with respect to the cell is increased at least about 5-fold, a least about 10-fold, at least about 20-fold, or more relative to a non-resistant cell from the same type of cancer.

In some embodiments, the MDR cancer cells express one or more ABC transporter proteins. Mechanisms of MDR include transporter-mediated resistance conferred by increased expression of the transmembrane glycoprotein, P-glycoprotein (Pgp), the product of the MDR1 gene and a related membrane glycoprotein, the multidrug resistance protein (MRP1). The mrp1 gene encodes a 190-kilodalton (kDa) transmembrane protein, whose structure is strikingly homologous to P-glycoprotein/MDR1 and other members of the ATP-binding cassette (ABC) transmembrane transporter proteins. There are at least five other human MRP isoforms identified. Among them, MRP2 (cMOAT) and MRP3 are also capable of supporting efflux detoxification of cancer drugs, including epipodophyllotoxins (MRP2 and 3), doxorubicin, and cisplatin (MRP2). MRP1, MRP2, MRP3 and MRP4 can all act as methotrexate efflux pumps and can confer resistance to methotrexate. Expression of these transporters can confer resistance to an overlapping array of structurally and functionally unrelated chemotherapeutic agents, toxic xenobiotics and natural product drugs. Cells in culture exhibiting MDR generally show reduced net drug accumulation and altered intracellular drug distribution. The sequence of P-glycoprotein may be obtained as Genbank accession number NM_{—}000927 (Chen et al. (1986) Cell 47:381-389.

In some embodiments of the invention, the cancer is assessed for its MDR status prior to treatment. Various methods are known in the art for determining whether a cell expressed an MDR transporter. In some such methods, the expression of an MDR gene is determined by quantitating the level of mRNA encoding the transporter by PCR, blot or array hybridization, in situ hybridization, and the like, as known in the art. In other embodiments, the presence of the transporter protein is directly determined, e.g. by immunoassays such as RIA, ELISA, immunohistochemistry, and the like.

In MDR1-expressing cells a decreased uptake of cytotoxic drugs can be visualized by measuring the cellular accumulation or uptake of fluorescent compounds, e.g., anthracyclines (Herweijer et al. (1989) Cytometry 10:463-468), verapamil-derivatives (Lelong et al. (1991) Mol. Pharmacol. 40:490-494), rhodamine 123 (Neyfakh (1988) Exp. Cell Res. 174:168-174); and Fluo-3 (Wall et al. (1993) Eur. J. Cancer 29:1024-1027). Alternatively, the sample of cells may be exposed to a calcein compound; measuring the amount of calcein compound accumulating in the specimen cells relative to control cells. Reduced calcein accumulation in specimen cells relative to control cells indicates the presence of multi-drug resistance in the biological specimen.

In other methods, imaging of the tumor is used to determine the functional presence of an MDR transporter. Noninvasive molecular imaging utilizes a transport substrate serving as a surrogate marker of chemotherapeutic agents. Compounds utilized in such assays include, without limitation, ^{99m}Tc-sestamibi, a widely available radiopharmaceutical that accumulates within cells in response to the physiologically negative mitochondrial and plasma membrane potentials, is approved as a tumor-imaging agent in breast, lung, thyroid, and brain cancers. Cellular accumulation of ^{99m}Tc-sestamibi into drug-sensitive tumor cells is high and translates into a “hot spot” on scintigraphic images or a slow washout rate from a tumor focus. However, functional MDR1Pgp mediates net outward transport of ^{99m}Tc-sestamibi from cells, thereby resulting in reduced net accumulation, detected either as a “cold” tumor or as a rapid washout rate from a tumor focus. Many studies have validated ^{99m}Tc-sestamibi for clinical analysis of MDR with imaging gamma cameras. Repetitive, noninvasive identification of transporter-mediated resistance can guide choices of chemotherapeutic agents.

[¹¹C]Verapamil is also a transport substrate for the Pgp efflux pump, and is being developed as a positron emission tomography (PET) agent for studying Pgp function non-invasively. Cellular accumulation of [¹¹C]verapamil correlates with Pgp expression, for example see Elsinga et al. (1996) J Nucl Med. 37(9):1571-1575; Hendrikse et al. (1999) Cancer Res. 59(10):2411-2416. 4-[¹⁸F]Fluoropaclitaxel is an alternative P-gp substrate useful in imaging (see Kurdziel et al. (2003) J Nuc/Med. 44(8):1330-1339).

^{99m}Tc-2-Methoxyisobutylisonitrile is another substrate of interest. Many clinical studies have been performed to correlate [^{99m}Tc]MIBI uptake or clearance with histological, molecular, and biochemical markers of various cellular processes, including apoptosis, proliferation, Pgp expression, and angiogenesis. The early tracer uptake reflects the mitochondrial status, which is affected by both apoptosis and proliferation. On the other hand, the tracer clearance reflects the activity of drug transporters such as Pgp. The uptake and clearance of [^{99m}Tc]MIBI by cancer cells may determine tumor response to anticancer treatment as shown in breast cancer, lung cancer, thyroid cancer, hepatocellular carcinoma, lymphoma and gastric cancer (see for example Del Vecchio (2004) Eur J Nucl Med Mol. Imaging. 31: S88-96. Suppl 1; Kawata et al. (2004) Clin Cancer Res. 10 (11):3788-93; Piwnica-Worms et al. (1995) Biochemistry. 34 (38):12210-20).

It will be understood by one of skill in the art that P-glycoprotein-associated MDR displays significant phenotypic heterogeneity. The relative degree of cross-resistance to drugs varies based on the cell line and the selecting drug. While the level of drug resistance is roughly correlated with the level of P-glycoprotein expression, protein and RNA levels may be disproportionately higher or lower than expected for the level of resistance observed. This phenotypic diversity may be the result of both MDR1 mutations and of posttranslational modifications of the MDR1 gene product.

P-glycoprotein RNA or protein has been detected in tumor specimens derived from patients with acute and chronic leukemias, ovarian cancer, multiple myeloma, breast cancer, neuroblastoma, soft tissue sarcomas, renal cell carcinoma, and others. Results have tended to link increased P-glycoprotein expression with a history of prior therapy or toxin exposure, and poorer treatment outcome. The relationship between increased P-glycoprotein and adverse outcome in human cancers is strongest in hematologic malignancies. Significant correlations between P-glycoprotein and adverse outcome in pediatric rhabdomyosarcoma and neuroblastoma have also been reported.

Chemotherapeutic agent. Agents that act to reduce cellular proliferation are known in the art and widely used. Agents of interest in the present invention include, without limitation, agents that are affected by transporter-mediated multidrug resistance. Such agents may include vinca alkyloids, taxanes, epipodophyllotoxins, anthracyclines, actinomycin, etc.

Anthracyclines are a class of chemotherapeutic agents based upon samine and tetra-hydro-naphthacene-dione. These compounds are used to treat a wide range of cancers, including (but not limited to) leukemias, lymphomas, and breast, uterine, ovarian, and lung cancers. Useful agents include daunorubicin hydrochloride (daunomycin, rubidomycin, cerubidine), doxorubicin, epirubicin, idarubicin, and mitoxantrone.

Vinca alkyloids are a class of drugs originally derived from the Vinca plant, and include vinblastine, vincristine, vindesine, vinorelbine. These agents bind tubulin, thereby inhibiting the assembly of microtubules.

Taxanes are diterpenes produced by the plants of the genus Taxus, and derivatives thereof. The principal mechanism of the taxane class of drugs is the disruption of microtubule function. It does this by stabilizing GDP-bound tubulin in the microtubule. The class includes paclitaxel and docetaxel.

Epipodophyllotoxins are naturally occurring alkaloids, and derivatives thereof. Epipodophyllotoxin derivatives currently used in the treatment of cancer include etoposide, teniposide.

Quinoline alkaloids are another class of interest. This class includes camptothecin, SN-38, DX-8951f, topotecan, 9-aminocamptothecin, BN 80915, irinotecan, DB 67, BNP 1350, exatecan, lurtotecan, ST 1481, and CKD 602.

Other natural products include azathioprine; brequinar; phenoxizone biscyclopeptides, e.g. dactinomycin; basic glycopeptides, e.g. bleomycin; anthraquinone glycosides, e.g. plicamycin (mithrmycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other chemotherapeutic agents include metal complexes, e.g. cisplatin (cis-DDP), carboplatin, etc.; ureas, e.g. hydroxyurea; and hydrazines, e.g. N-methylhydrazine. Retinoids, e.g. vitamin A, 13-cis-retinoic acid, trans-retinoic acid, isotretinoin, etc.; carotenoids, e.g. beta-carotene, vitamin D, etc. Retinoids regulate epithelial cell differentiation and proliferation, and are used in both treatment and prophylaxis of epithelial hyperproliferative disorders.

The terms “guanidyl,” “guanidinyl” and “guanidino” are used interchangeably to refer to a moiety having the formula —HN═C(NH₂)NH (unprotonated form). As an example, arginine contains a guanidyl (guanidino) moiety, and is also referred to as 2-amino-5-guanidinovaleric acid or α-amino-β-guanidinovaleric acid. “Guanidium” refers to the positively charged conjugate acid form. The term “guanidino moiety” includes, for example, guanidine, guanidinium, guanidine derivatives such as (RNHC(NH)NHR′), monosubstituted guanidines, monoguanides, biguanides, biguanide derivatives such as (RNHC(NH)NHC(NH)NHR′), and the like. In addition, the term “guanidino moiety” encompasses any one or more of a guanide alone or a combination of different guanides.

“Amidinyl” and “amidino” refer to a moiety having the formula —C(═NH)(NH₂). “Amidinium” refers to the positively charged conjugate acid form.

The compounds of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intracheal, intravenous, etc., administration. The active agent may be systemic after administration or may be localized by the use of regional administration, intramural administration, or use of an implant that acts to retain the active dose at the site of implantation, or combination of the above.

In pharmaceutical dosage forms, the compounds may be administered in the form of their pharmaceutically acceptable salts. They may also be used in appropriate association with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier.

Implants for sustained release formulations are well-known in the art. Implants are formulated as microspheres, slabs, etc. with biodegradable or non-biodegradable polymers. For example, polymers of lactic acid and/or glycolic acid form an erodible polymer that is well-tolerated by the host. The implant containing the therapeutic agent is placed in proximity to the site of the tumor, so that the local concentration of active agent is increased relative to the rest of the body.

The term “unit dosage form”, as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the unit dosage forms of the present invention depend on the particular compound employed and the effect to be achieved, and the pharmacodynamics associated with each compound in the host.

Pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

The host, or patient, may be from any mammalian species, e.g. primate sp., particularly humans; rodents, including mice, rats and hamsters; rabbits; equines, bovines, canines, felines; etc. Animal models are of interest for experimental investigations, providing a model for treatment of human disease.

Cancers of interest include carcinomas, e.g. colon, prostate, breast, melanoma, ductal, endometrial, stomach, dysplastic oral mucosa, invasive oral cancer, non-small cell lung carcinoma, transitional and squamous cell urinary carcinoma, etc.; neurological malignancies, e.g. neuroblastoma, gliomas, etc.; hematological malignancies, e.g. childhood acute leukemia, non-Hodgkin's lymphomas, and other myeloproliferative disorders, chronic lymphocytic leukemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; and the like. Cancers of interest particularly include hematologic cancers, e.g. acute myelogenous leukemia, chronic myelogenous leukemia, acute lymphocytic leukemia, Hodgkin's lymphoma, non-Hodgkin's lymphoma, multiple myeloma, etc.; ovarian cancer; breast cancer; neuroblastoma; soft tissue sarcomas; renal cell carcinoma, all of which are have a high tendency to develop multidrug resistance.

The majority of breast cancers are adenocarcinomas subtypes. Ductal carcinoma in situ is the most common type of noninvasive breast cancer. In DCIS, the malignant cells have not metastasized through the walls of the ducts into the fatty tissue of the breast. Infiltrating (or invasive) ductal carcinoma (IDC) has metastasized through the wall of the duct and invaded the fatty tissue of the breast. Infiltrating (or invasive) lobular carcinoma (ILC) is similar to IDC, in that it has the potential metastasize elsewhere in the body. About 10% to 15% of invasive breast cancers are invasive lobular carcinomas.

Ovarian cancer is often fatal because it is usually advanced when diagnosed. Symptoms are usually absent in early stage and nonspecific in advanced stage. Evaluation usually includes ultrasonography, CT or MRI, and measurement of tumor markers, e.g. CA125. Diagnosis is by histologic analysis. Staging is surgical. In the US, ovarian cancer is the 2nd most common gynecologic cancer and the deadliest; it is the 5th leading cause of cancer-related deaths in women.

Ovarian cancers are histologically diverse. At least 80% originate in the epithelium; 75% of these cancers are serous cystadenocarcinoma, and the rest include mucinous, endometrioid, transitional cell, clear cell, unclassified carcinomas, and Brenner tumor. The remaining 20% of ovarian cancers originate in primary ovarian germ cells or in sex cord and stromal cells or are metastases to the ovary (most commonly, from the breast or GI tract). Germ cell cancers usually occur in women <30 and include dysgerminomas, immature teratomas, endodermal sinus tumors, embryonal carcinomas, choriocarcinomas, and polyembryomas. Stromal (sex cord—stromal) cancers include granulosa-theca cell tumors and Sertoli-Leydig cell tumors.

Intraperitoneally (IP) injected taxol has shown promising clinical results for the treatment of ovarian cancer including significant life extensions, prompting a recent NCl clinical announcement on the merit of this procedure (Armstrong D K, et al. (2006) N Engl J Med 354:34-43.). IP-administered taxol allows drug delivery directly to targeted tissue, thereby minimizing systemic exposure and off target side effects. However, because taxol is poorly water soluble (˜0.4 μg/mL), it must still be formulated with Cremophor EL, a vehicle that often elicits hypersensitivity side effects and because of the formulated volume requires extended administration times. In contrast, oligoarginine conjugates of taxol are freely water soluble, thus precluding the need for Cremophor and allowing for smaller administration volumes and thus shorter administration times.

Neuroblastoma is a cancer arising in the adrenal gland or less often from the extra-adrenal sympathetic chain, including the retroperitoneum, chest, and neck. Diagnosis is based on biopsy. Treatment may include surgical resection, chemotherapy, radiation therapy, and high-dose chemotherapy with stem cell transplantation. Neuroblastoma is the most common cancer in infants. Almost 90% of cases present in children <5 yr. There are numerous different cytogenetic abnormalities on several chromosomes that can result in neuroblastoma; in 1 to 2%, abnormalities appear to be inherited. Some markers (eg, N-myc oncogene, hyperdiploidy) correlate with progression and prognosis.

Renal cell carcinoma (RCC), an adenocarcinoma, accounts for 90 to 95% of primary malignant renal tumors. Symptoms appear late and include hematuria, flank pain, a palpable mass, and FUO. Diagnosis is by CT or MRI and occasionally by biopsy. Treatment is with surgery for early disease and typically an experimental protocol for advanced disease.

In some embodiments of the invention, the cancer being treated comprises cancer stem cells. Optionally, the tumor is tested for the presence of cancer stem cells, or proportion of cancer stem cells in the population is determined. Cancer stem cells may have inherently high degree of efflux mechanisms that mediate resistance to chemotherapeutic agents.

The term “cancer stem cells,” as defined herein, refers to a subpopulation of tumorigenic cancer cells with both self-renewal and differentiation capacity. These tumorigenic cells are responsible for tumor maintenance and also give rise to large numbers of abnormally differentiating progeny that are not tumorigenic. These cancer stem cells form tumors in vivo; self-renew to generate tumorigenic progeny; give rise to abnormally differentiated, nontumorigenic progeny, and differentially express at least one stem cell-associated gene.

Certain phenotypic attributes of carcinoma stem cells have been described in the art, and may include markers such as CD44, CD133, CD24, CD49f; ESA; CD166; and lineage panels. Examples of specific marker combinations and phenotypes are described, for example, by Al-Hajj et al. (2003) Prospective identification of tumorigenic breast cancer cells. Proc Natl Acad Sci USA 100, 3983-8; Singh et al. (2004) Identification of human brain tumour initiating cells. Nature 432, 396-401; Dalerba et al. (2007) Phenotypic characterization of human colorectal cancer stem cells. Proc Natl Acad Sci USA 104, 10158-63; O'Brien et al. (2006) A human colon cancer cell capable of initiating tumour growth in immunodeficient mice. Nature; Prince et al. (2007) Identification of a subpopulation of cells with cancer stem cell properties in head and neck squamous cell carcinoma. Proc Natl Acad Sci USA, each of which is herein specifically incorporated by reference for the teachings of cancer stem cell marker phenotypes. In some embodiments of the invention such phenotyping is used in conjunction with the cancer treatment.

The present invention provides compositions and methods that enhance the therapeutic efficacy of chemotherapeutic drugs in the treatment of multidrug resistant cancers. The methods involve contacting the cancer cells with a conjugate that includes the chemotherapeutic drug linked to a delivery-enhancing transporter. The delivery enhancing transporters are molecules that include sufficient guanidino or amidino moieties to increase delivery of the conjugate into the cancer cell.

The transporter moiety could be any of the molecular transports defined above. In some cases, peptidic transporter moiety comprises at least 5 guanidino and/or amidino moieties, and more preferably 7 or more such moieties. Preferably, the delivery-enhancing transporters have 25 or fewer guanidino and/or amidino moieties, and often have 15 or fewer of such moieties. The delivery-enhancing transporter can be as short as 5 subunits, in which case all subunits include a guanidino or amidino sidechain moiety. The delivery-enhancing transporters can have, for example, at least 6 subunits, and in some embodiments have at least 7, 8, 9 or 10 subunits. Generally, at least 50% of the subunits contain a guanidino or amidino sidechain moiety. More preferably, at least 70% of the subunits, and sometimes at least 90% of the subunits in the delivery-enhancing transporter contain a guanidino or amidino sidechain moiety.

Some or all of the guanidino and/or amidino moieties in the delivery-enhancing transporters can be contiguous. For example, the transporter moiety can include from 5 to 25 contiguous guanidino and/or amidino-containing subunits. Six, seven, eight or more contiguous guanidino and/or amidino-containing subunits are present in some embodiments. In some embodiments, each subunit that contains a guanidino moiety is contiguous, as exemplified by a polymer containing at least six, at least seven, at least eight, and not more than twelve contiguous arginine residues.

Such an arginine-containing peptide can be composed of either all D-, all L- or mixed D- and L-amino acids, and can include additional amino acids, amino acid analogs, or other molecules between the arginine residues. The transporter may also have a non-peptidic backbone, e.g. peptoid, oligocarbamate, polyamines, polysaccharides, steroids, cationic lipids, guanidinoglycosides, and even nanotubes. In addition, many of these transporters can be hybridized (e.g., steroid-modified oligoguanidines) to create new transporter types that could be used in the invention. In some embodiments the delivery enhancing transporter is a classical CPP. Examples include Tat 9-mer (RKKRRQRRR or Tat49-57), transportan, penetration, antennapedia and derivatives of thereof. Optionally, the transporter conjugate includes a linker, for example a disulfide linker as described herein. The use of at least five D-arginine in the delivery-enhancing transporters can enhance biological stability of the transporter during transit of the conjugate to its biological target. In some cases the delivery-enhancing transporters are at least about 50% D-arginine residues, or all of the subunits are D-arginine residues.

The transporter moiety may be constructed by any method known in the art. Exemplary peptide polymers can be produced synthetically, preferably using a peptide synthesizer (e.g., an Applied Biosystems Model 433) or can be synthesized recombinantly by methods well known in the art. Recombinant synthesis is generally used when the delivery enhancing transporter is a peptide which is fused to a polypeptide or protein of interest. Peptides are generally produced with an amino terminal protecting group, such as FMOC. For chemotherapeutic drugs that can survive the conditions used to cleave the polypeptide from the synthesis resin and deprotect the sidechains, the FMOC may be cleaved from the N-terminus of the completed resin-bound polypeptide so that the agent can be linked to the free N-terminal amine. In such cases, the chemotherapeutic drug to be attached is typically activated by methods well known in the art to produce an active ester or active carbonate moiety effective to form an amide or carbamate linkage, respectively, with the polymer amino group. In the examples provided herein, the thiopyridyl moiety of the linker already attached to a drug (see Methods section) was displaced with free thiol of acylated D-cysteine D-octaarginine (AcNHcr8CONH₂) to give the transporter-linker conjugate. Of course, other linking chemistries can also be used.

N-methyl and hydroxy-amino acids can be substituted for conventional amino acids in solid phase peptide synthesis. However, production of delivery-enhancing transporters with reduced peptide bonds requires synthesis of the dimer of amino acids containing the reduced peptide bond. Such dimers are incorporated into polymers using standard solid phase synthesis procedures, or by using scalable solution-phase synthesis on the basis of a segment doubling strategy (Wender P A, et al. (2001) Org Lett 3:3229-3232.) Other synthesis procedures are well known and can be found, for example, in Fletcher et al. (1998) Chem. Rev. 98:763, Simon et al. (1992) Proc. Nat'l. Acad. Sci. USA 89:9367, and references cited therein.

The chemotherapeutic drug can be linked to the transporter moiety according to a number of embodiments. In one embodiment, the agent is linked to a single delivery-enhancing transporter, either via linkage to a terminal end of the delivery-enhancing transporter or to an internal subunit within the reagent via a suitable linking group. The agent is generally not attached to one any of the guanidino or amidino sidechains so that they are free to interact with the target membrane. The conjugates of the invention can be prepared by straightforward synthetic schemes. Furthermore, the conjugate products are usually substantially homogeneous in length and composition, so that they provide greater consistency and reproducibility in their effects than heterogeneous mixtures.

Suitable linkers are known in the art (see, for example, Wong, S. S., Ed., Chemistry of Protein Conjugation and Cross-Linking, CRC Press, Inc., Boca Raton, Fla. (1991). In particular, carbamate, ester, thioether, disulfide, and hydrazone linkages are generally easy to form and suitable for most applications. Other linkers such as trimethyl lock (see Wang et. al. J. Org. Chem., 62:1363 (1997) and Chandran et al., J. Am. Chem. Soc., 127:1652 (2005)), quinine methide linker (see Greenwald et. al. J. Med. Chem., 42:3657 (1999) and Greenwald et. al. Bioconjugate Chem., 14:395 (2003)), diketopiperazine linker and derivatives of thereof are also of interest of this invention.

Ester and disulfide linkages are preferred if the linkage is to be readily degraded in a biological environment, after transport of the substance across the cell membrane. Ester linkers can also be cleaved extracellularly with the help of extracellular esterases. Various functional groups (hydroxyl, amino, halogen, thiol etc.) can be used to attach the chemotherapeutic drug to the transport polymer or to a linker, incorporated between a drug and a transporter. Groups which are not known to be part of an active site of the biologically active agent are preferred, particularly if the polypeptide or any portion thereof is to remain attached to the substance after delivery. Releasable linkers could be used if the attachment is done at the site of molecule important for biological activity.

To help minimize side-reactions, guanidino and amidino moieties can be blocked using conventional protecting groups, such as carbobenzyloxy groups (CBZ), di-t-BOC, PMC, Pbf, N—NO₂, and the like.

Coupling reactions are performed by known coupling methods in any of an array of solvents, such as N,N-dimethyl formamide (DMF), N-methylpyrrolidinone, dichloromethane, water, and the like. Exemplary coupling reagents include, for example, O-benzotriazolyloxy tetramethyluronium hexafluorophosphate (HATU), dicyclohexyl carbodiimide, bromotris(pyrrolidino) phosphonium bromide (PyBroP), etc. Other reagents can be included, such as N,N-dimethylamino pyridine (DMAP), 4-pyrrolidino pyridine, N-hydroxy succinimide, N-hydroxy benzotriazole, and the like.

The chemotherapeutic drugs are usually attached to the transporter moiety using a linkage that is specifically cleavable or releasable. The use of such linkages is particularly important for chemotherapeutic drugs that are inactive until the attached transporter moiety is released. In some cases, such conjugates can be referred to as prodrugs, in that the release of the delivery-enhancing transporter from the drug results in conversion of the drug from an inactive to an active form. As used herein, “cleaved” or “cleavage” of a conjugate or linker refers to release of a chemotherapeutic drugs from a transporter moiety, thereby releasing an active chemotherapeutic drugs. “Specifically cleavable” or “specifically releasable” refers to the linkage between the transporter and the drug being cleaved, rather than the transporter being degraded (e.g., by proteolytic degradation). However, this “degradable” mechanism of drug release could also be used in the invention.

In some embodiments, the linkage is a readily cleavable linkage, meaning that it is susceptible to cleavage under conditions found in vivo. Thus, upon passing into a cancer cell the drug is released from the transporter. Readily cleavable linkages can be, for example, linkages that are cleaved by an enzyme having a specific activity (e.g., an esterase, protease, phosphatase, peptidase, and the like) or by hydrolysis. For this purpose, linkers containing carboxylic acid esters and disulfide bonds are sometimes preferred, where the former groups are hydrolyzed enzymatically or chemically, and the latter are severed by disulfide exchange, e.g., in the presence of glutathione. The thiol resulting from glutathione cleavage was expected to cyclize into the proximate carbonyl group of the linker, leading subsequently to the release of free drug at a rate controlled by linker design.

In some embodiments the conjugate has the structure shown in Formula I

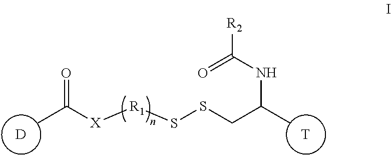

where X is CH₂; C(CH₃)₂; 0; NH; or S;
R₁ is CH₂; C(CH₃)₂; C(C₂H₅)₂, or a combination thereof;
R₂ is CH₃, any alkyl chain, e.g. a C₁-C₆ lower alkyl, amino acid or peptide;
n=0-5;
D is a chemotherapeutic drug; and
T is a transporter moiety, usually linked to an amine.

A conjugate in which a drug to be delivered and a transporter are linked by a specifically cleavable or specifically releasable linker will have a half-life. The term “half-life” in this context refers to the amount of time required for one half of the amount of conjugate to become dissociated to release the free drug. The “accelerated” (37° C.) hydrolytic half-lives of the conjugates ranged from 19 to 97 hours, extending well beyond the incubation times (≦20 minutes) used for cell assays. Stabilities of the conjugates as solids at room temperature extend for months. In contrast, cleavage of the disulfide linker in the presence of dithiothreitol (analog of glutathione) occurred in seconds for all compounds and resulted in the subsequent release of free drug (half-lives indicated in Table 1). The half-life of a conjugate can be “tuned” or modified, according to the invention, as described below.

### Methods of Use

The compounds of the invention have been shown to have anti-proliferative effect in an in vivo xenograft tumor model. The present compounds are useful for prophylactic or therapeutic purposes. As used herein, the term “treating” is used to refer to both prevention of disease, and treatment of pre-existing conditions. The prevention of proliferation is accomplished by administration of the subject compounds prior to development of overt disease, e.g., to prevent the regrowth of tumors, prevent metastatic growth, etc. Alternatively the compounds are used to treat ongoing disease, by stabilizing or improving the clinical symptoms of the patient.

The host, or patient, may be from any mammalian species, e.g., primate sp., particularly humans; rodents, including mice, rats and hamsters; rabbits; equines, bovines, canines, felines; etc. Animal models are of interest for experimental investigations, providing a model for treatment of human disease.

The susceptibility of a particular cell to treatment with the subject compounds may be determined by in vitro testing. Typically a culture of the cell is combined with a subject compound at varying concentrations for a period of time sufficient to allow the active agents to induce cell death or inhibit migration, usually between about one hour and one week. For in vitro testing, cultured cells from a biopsy sample may be used. The viable cells left after treatment are then counted.

The dose will vary depending on the specific compound utilized, specific disorder, patient status, etc. Typically a therapeutic dose will be sufficient to substantially decrease the undesirable cell population in the targeted tissue, while maintaining patient viability. Treatment will generally be continued until there is a substantial reduction, e.g., at least about 50%, decrease in the cell burden, and may be continued until there are essentially none of the undesirable cells detected in the body.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “a cell” includes a plurality of such cells and reference to “the array” includes reference to one or more arrays and equivalents thereof known to those skilled in the art, and so forth. All technical and scientific terms used herein have the same meaning as commonly understood to one of ordinary skill in the art to which this invention belongs unless clearly indicated otherwise.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### Example 1

### Overcoming Multi-Drug Resistance and Improving Efficacy and Solubility Through Conjugation of Small Molecules to Octaarginine Transporters

Oligoarginine molecular transporters are highly charged cell penetrating peptides that can be attached to a drug or probe cargo to produce conjugates often exhibiting improved aqueous solubility, cellular and tissue uptake, selectivity, and efficacy relative to the cargo alone. Since small molecules or drugs conjugated to oligoarginine transporters enter cells via a mechanism different from passive diffusion oligoarginine transporter conjugates also offer a means of overcoming off-target effects such as the efflux of therapeutic agents by proteins involved in multidrug resistance. Here we show that conjugation of oligoarginine peptides to a representative small, therapeutic molecule (taxol) can modify its in vivo distribution, improve its solubility and pharmacokinetic properties, and significantly improve activity against malignant cells otherwise resistant to the therapeutic agent alone.

The anticancer agent taxol (paclitaxel, 1) has revolutionized the treatment of cancer and markedly improved the survival of patients. However, despite the hope and promise that taxoids have engendered, their lack of activity against MDR tumors as well as dose-limiting toxicity are significant limitations. Additionally, taxol and many of its derivatives exhibit poor aqueous solubility due to their hydrophobic nature, and thus require prolonged intravenous administration, often placing a further demand on the treated patient. Taxol itself, for example, has only poor solubility in water (˜0.4 μg/mL) and must be dissolved in Cremophor EL at low concentrations for intravenous administration. Significant side effects associated with hypersensitivity to Cremophor EL have been observed and the formulation could even reduce the antitumor efficacy of the administered drug. These problems associated with taxol are not uncommon and are associated with many other therapeutic agents and drug candidates. For example, camptothecin, a promising topoisomerase inhibitor lead, has failed to advance because of its poor aqueous solubility.

To explore the effect of oligoarginine transporter-assisted delivery of small molecule therapeutics on cancer cells sensitive or resistant to the therapeutic agent alone, we set out to attach an octaarginine transporter to the C2′ or C7 positions of taxol using a biocleavable disulfide linker (compounds 2-4, Table 1).

TABLE 1
Structures, stability and release kinetics of octaarginine-drug/probe conjugates used in the study.

| | | DTT^{a} | HBS^{b} |
|---|---|---|---|
| 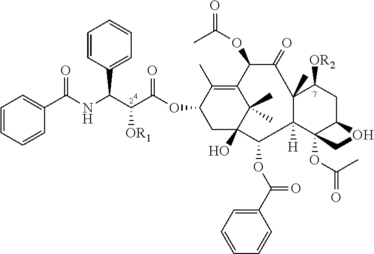 | 1: R₁ = R₂ = H (Taxol) 2a (n = 8); 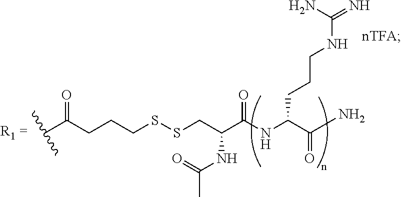 R₂ = H 2b (n = 4) 3a (n = 8); R₁ = H; 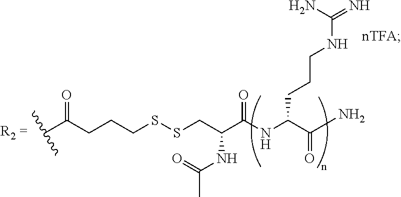 3b (n = 4) 4a (n = 8); 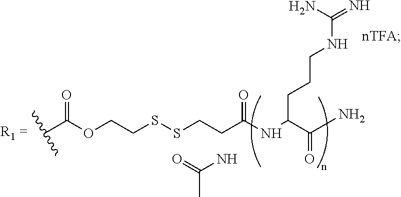 R₂ = H 4b (n = 4) | — 3 min 3 min 1 hr 1 hr 6 min 5 min | — 23 hr 19 hr 97 hr 68 hr 32 hr 26 hr |
| 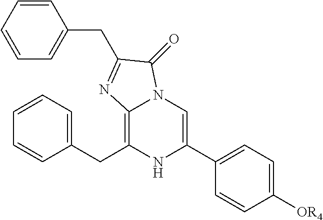 | 5: R₄ = H (coelenterazine H); 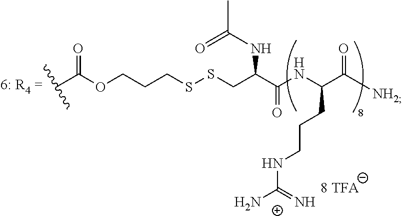 | <1 min | 7 min |
| 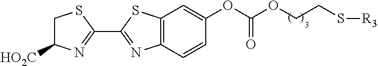 | 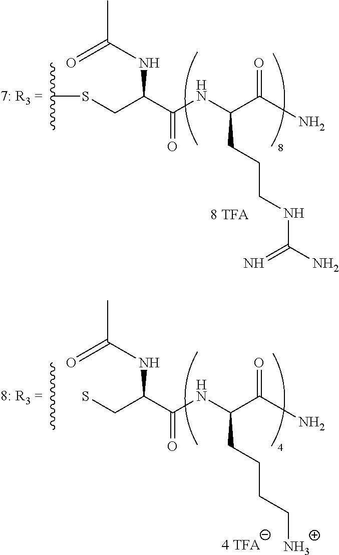 | 3 min | 30 hr |

^{a}Stability in reducing environment (10 mM dithiothreitol (DDT), HBS, pH = 7.4, 37° C.);
^{b}hydrolytic stabililty (HBS, pH = 7.4, 37° C.).

An octaarginine transporter was selected because of its demonstrated ability to enhance cellular uptake and its ease of synthesis. Preference was also given to a disulfide linker because its cleavage would occur only after cellular entry of the conjugate upon encountering a high glutathione concentration (typically 15 mM intracellular compared to 15 μM extracellular) and at a rate controlled by linker design. The thiol resulting from glutathione cleavage was expected to cyclize into the proximate carbonyl group of the linker, leading subsequently to the release of free taxol. The position of attachment of the linker to taxol and the linking functionality were expected to be important for activity in both taxol-sensitive and taxol-resistant cell lines. Because modification of the C2′ alcohol of taxol is known to result in considerable loss of activity, (Kingston (2000) Journal of Natural Products 63, 726-734) we set out to make one class of conjugates with a linker at C2′ (compounds 2a-b and 4a-b) that would be effective in cells only if the free drug were released. Additionally, because the C7 position of taxol can be modified without significant loss in activity and is important in the interaction of taxanes with Pgp, we also prepared a class of conjugates attached to C7 via the same disulfide releasable linker (compounds 3a-b).

TABLE 2
IC₅₀ concentrations (nM)^{a} of taxol and its octaarginine^{b}
conjugates in selected cancer cell lines.

| | | | | C2′ r8 |
|---|---|---|---|---|
| | Taxol | C2′ ester r8 | C7 ester r8 | carbonate |
| Cell line | (1)^{c} | (2a)^{d} | (3a)^{d} | (4a)^{d} |
| UCI-101 luc | 246 | 58 | 170 | 95 |
| UCI-107 luc | 320 | 92 | 233 | 144 |
| SKOV-3 | 29 | 11 | 24 | 18 |
| OVCA 429 | 147 | 42 | 125 | 77 |
| OVCA 429T | 15802 | 420 | 225 | 781 |
| OVCA 429TxT | 2521 | 101 | 93 | 216 |
| OVCA 433 | 294 | 21 | 62 | 44 |
| OVCA 433T | 6183 | 131 | 95 | 207 |
| OVCA 433TxT | 12365 | 2977 | 1280 | 7853 |
| MCF-7 | 171 | 40 | 146 | 108 |
| MCF-7-pgp | 867 | 151 | 83 | 230 |

^{a}IC₅₀ concentrations were determined by incubating the cells with the compound for 20 min, washing them twice with fresh media, followed by a 72 hr incubation in drug-free media. Viability was then measured by MTT assay or luciferase activity.
^{b}Data for tetraarginine analogs (2b-4b) are not shown because none of them displayed activity in this assay (IC₅₀ >> 10 μM).
^{c}Taxol was administered in 2% DMSO solution in PBS.
^{d}Conjugates were administered in 100% PBS.

Tetraarginine transporter conjugates were used as negative controls because they possess all of the features of the octaarginine conjugates, including aqueous solubility and linking functionality, but they do not readily enter cells and thus would only exhibit activity if they cleaved extracellularly to produce free drug. The “accelerated” (37° C.) hydrolytic half-lives of the tetra- and octaarginine conjugates ranged from 19 to 97 hours, extending well beyond the incubation times (s 20 minutes) used for cell assays. Stability as a solid at room temperature extends for months. In contrast, cleavage of the disulfide linker (in the presence of dithiothreitol) occurred in seconds for all compounds and resulted in the subsequent release of free drug (half-lives indicated in Table 1). The activities of various conjugate-linker combinations were evaluated using a panel of human cancer cells, leading to the identification of ester-linked conjugates 2a and 3a as preferred candidates (Table 2). Conjugates with a carbonate based releasable disulfide linker (4) were significantly less active in cells (Table 2), while conjugates with longer ester and carbonate linkers did not show activity.

Cells from a panel of cancer cell lines (Table 2) were incubated for 20 minutes with taxol or octa- or tetraarginine (r8 and r4) taxol conjugates and washed to remove any remaining agent that did not enter cells. After 72 hours, the cytotoxicity of the relevant compound was determined by an MTT based assay. Cell killing (expressed as IC₅₀ values; the concentration at which the viability of the cells in culture is reduced by 50%) mediated by several of the octaarginine conjugates was significantly better than that observed for free taxol administered in DMSO or water. Only a twenty minute exposure was required to differentiate the activities of taxol and taxol conjugates used in equimolar concentrations. No extracellular hydrolytic release of taxol from the conjugate occurred during the incubation period as evident from the dramatically different activities of the conjugate and the free drug and the lack of activity of the tetraarginine control conjugates (2b-4-b) which, while similar in functionality to the octaarginine conjugates, do not readily enter cells.

Within the panel of human cancer cells, there were three sets of related sensitive and resistant cell lines, the latter based wholly or in part on Pgp-mediated efflux. These resistant lines were either created through stable transfection of MCF-7 cells with Pgp (MCF-7-Pgp), or by selection through exposure of OVCA429 and OVCA433 cells to taxol (OVCA429T and OVCA433T)¹⁹ or taxotere (OVCA429TxT and OVCA433TxT), creating a complex MDR phenotype including Pgp upregulation. Remarkably, for all resistant cells, taxol conjugates 2a and 3a were both more effective than taxol itself, and both displayed an ability to overcome drug resistance, with the C7 analog 3a consistently being more effective.

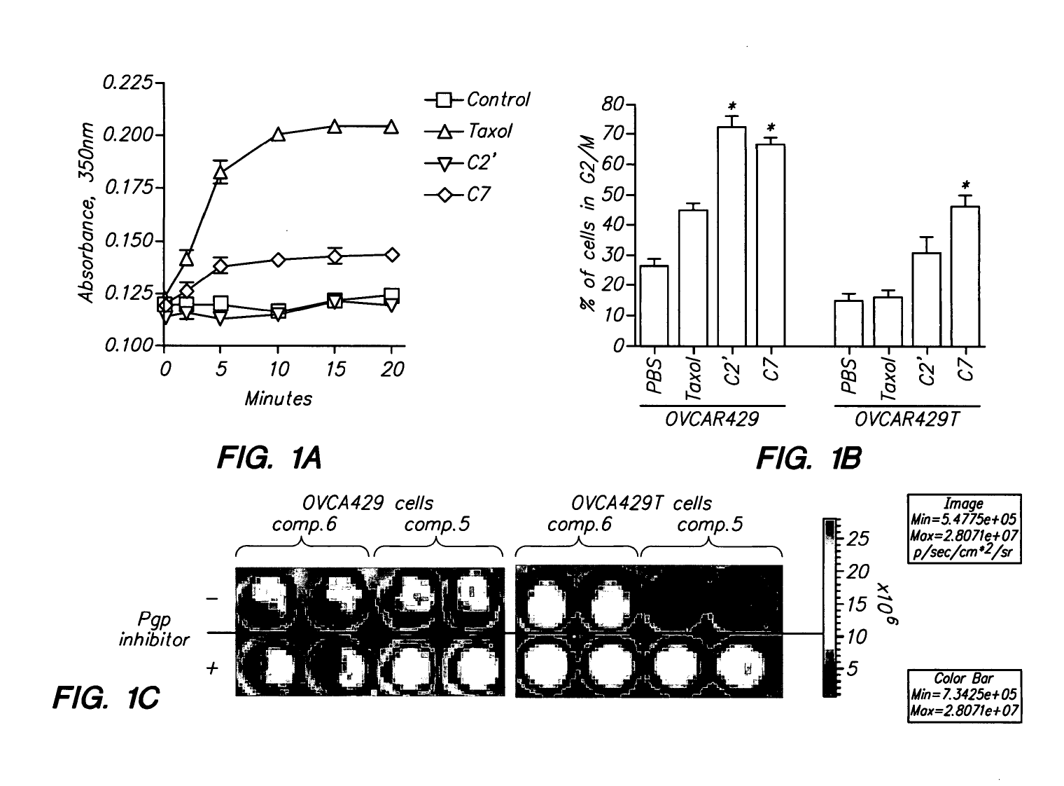

To determine whether the mechanism of action of the r8 conjugated taxols paralleled that of taxol, in vitro (cell free) tubulin depolymerization and cell cycle assays were conducted (FIG. 1). As expected, in the tubulin depolymerization assay (FIG. 1a), only taxol was active, indicating that the r8 conjugates are stable in the absence of disulfide-cleaving agents. A cell cycle assay was also conducted, showing that the r8 conjugates were killing tumor cells through the same cell cycle arrest mechanism as taxol (FIG. 1b). As expected, conditions and conjugates that produced the greatest loss of cell viability correlated with increased accumulation of cells in the G2/M interphase. Both C2′ and C7 r8 conjugates (2a and 2b) produced a significantly greater percentage of OVCA429 cells in the G2/M phase than taxol (p=0.0045 for C2′ and 0.0051 for C7).

It was also seen that the killing of the Pgp-upregulated MDR cell line OVCA429T by the r8 conjugates was similarly due to a block of cell cycle entry into M-phase. Relative to taxol, only the C7 conjugate produced a significantly greater number of resistant cells in G2/M arrest (p=0.0034 for C7; p=0.073 for C2′). The activity of this conjugate against resistant cells was as effective as the activity of taxol against taxol-sensitive cells (OVCA429), indicating that r8 conjugation to the C7 position of taxol is capable of completely overcoming the MDR phenotype in this cell line. The effect of the oligoarginine transporter was further examined using the octaarginine conjugate (6) of coelenterazine H (5), the latter a substrate for Renilla luciferase and, like taxol, a substrate for Pgp-mediated efflux. Coelenterazines are made up of a lipophilic, amine-containing heterocycle, with physicochemical properties similar to other substrates of Pgp. As expected, when coelenterazine H was incubated with multidrug resistant OVCA429T cells transfected with Renilla luciferase, the bioluminescence signal was reduced relative to the signal obtained with OVCA429 cells (FIG. 1c).

Significantly, the r8 conjugate of coelenterazine H (6) overcame this efflux mediated signal reduction, exhibiting similar luminescence in both cell lines. Preincubation of OVCA429T cells with the Pgp inhibitor cyclosporine A also overcame the signal reduction observed for coelenterazine H (5), but had little effect on the r8 conjugate treated OVCA429T cells, indicating that these effects are indeed Pgp mediated. The ability of the r8 transporter to circumvent Pgp-mediated efflux of such varied structures (i.e., 1 and 5) suggests that this approach could be extended to other small molecule drugs and leads.

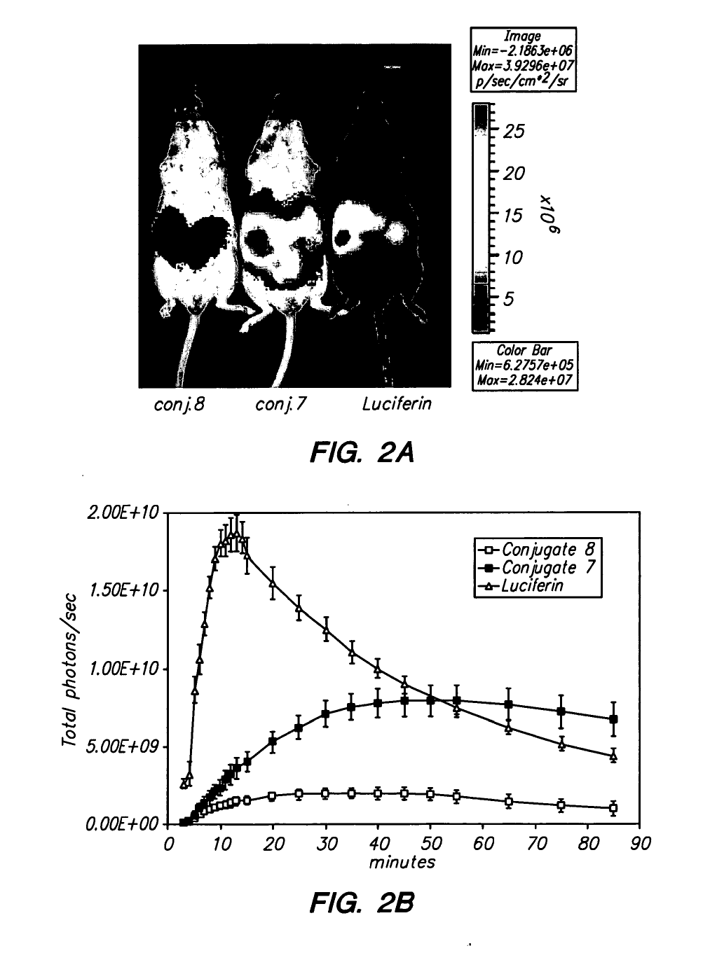

As a prelude to animal studies with taxol conjugates, a third small molecule system, luciferin, the substrate for Firefly luciferase, was used as a drug surrogate to compare the biodistribution and pharmacokinetics of the r8 drug surrogate conjugate and the surrogate alone. Luciferin and luciferin conjugated to r8 (7) were delivered via intraperitoneal injection into transgenic mice ubiquitously expressing Firefly luciferase (FIG. 2) at concentrations equimolar to those used in the studies with taxol (5 mg/kg). Because of the adherence and rapid cellular uptake of the r8 conjugate, it did not distribute throughout the body, remaining instead near the site of administration. For localized tumors, this transporter based effect allows for greater accumulation of drug in the vicinity of the tumor and correspondingly reduced systemic toxicity.

Ovarian cancer with intraperitoneal drug administration was chosen for this work based in part on the recent recommendation of the NCl that approved treatments for advanced ovarian cancer (taxol, cisplatin, carboplatin) include intraperitoneal delivery. In addition, although uptake of the conjugate is rapid, the sustained rate of release of the free drug allows for the maintenance of constant and controlled drug levels thereby avoiding the bolus effect encountered when a free drug is administered. Pertinent to this point, comparison of the bioluminescence resulting from the r8 luciferin conjugate 7 and from luciferin alone showed that the latter produced a signal that peaked early (about 12 minutes post injection) and declined rapidly (FIG. 2b). The calculated area under the curve (total light production) was similar for both r8 conjugate 7 and luciferin alone, demonstrating that the drug surrogate (luciferin) was released efficiently from the conjugate but at a level sustained over time. The tetralysine luciferin conjugate 8, possessing the same disulfide linker and cargo and similar polycation based water solubility as the r8 conjugate, was used as a control.

As expected, the k4 conjugate 8 produced 3.8 fold less total signal, confirming the important role of the enhanced cellular uptake of the octaarginine transporter in the activity of its conjugates. Overall, the taxol conjugates are readily administered in aqueous solution thereby avoiding prolonged administration of taxol using Cremophor EL, remain localized due to cell adherence and the rapid rate of cellular uptake and, due to the sustained release of the free drug, avoid or minimize adverse peak-trough effects arising from a bolus injection.

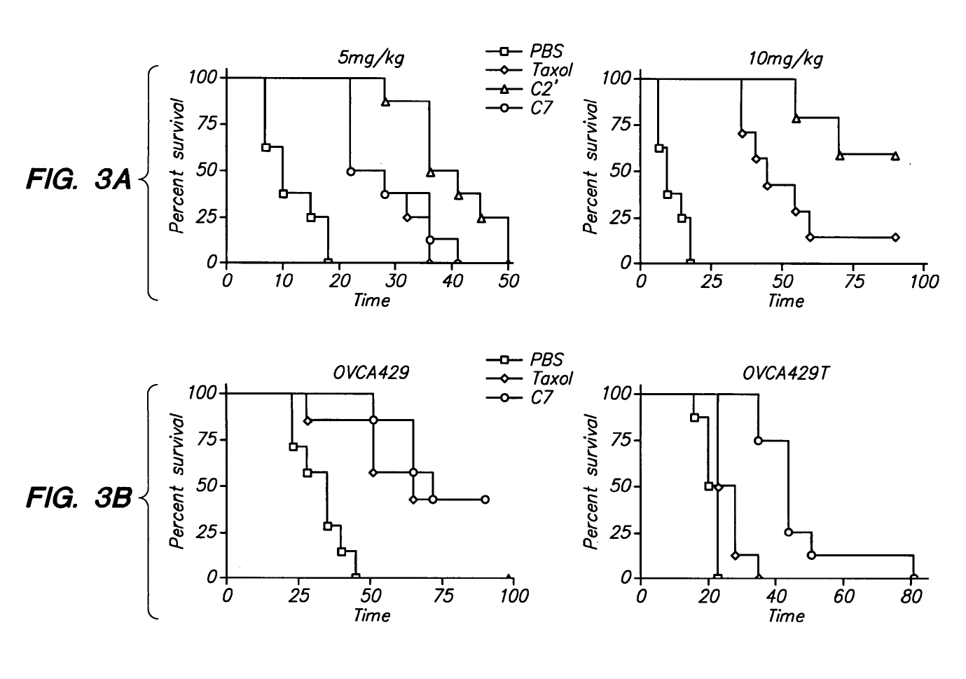

Several different mouse tumor models of ovarian cancer were examined next (FIG. 3). In an initial study the human ovarian tumor cell line UCI-101 expressing luciferase was implanted into the peritoneal cavity. The animal was then treated with doses of 5 or 10 mg/kg (5 mg/kg is equivalent to the clinical dose of taxol recommended to treat ovarian cancer) of either taxol (administered in 10% DMSO PBS solution) or C2′ r8 taxol (2a) (administered in PBS) delivered via intraperitoneal injection. At both doses the r8 conjugate produced enhanced anticancer effects over taxol alone (p=0.0039 at 5 mg/kg and 0.047 at 10 mg/kg), with the 10 mg/kg dose resulting in 60% complete responses (compared to 12.5% complete responses for animals treated with taxol at the same dose). While the C7 conjugated taxol (3a) was found to be only as effective as taxol against taxol-sensitive tumors, mirroring the cell line cytotoxicity assays, it was shown to overcome taxol resistance in cells with Pgp-mediated multidrug resistance phenotypes (Table 2). This was also found to be the case in the animal models, for mice bearing peritoneal tumors formed from OVCA429 (taxol sensitive) or OVCA429T (taxol resistant); whereas both C7 conjugate (3a) (administered in PBS) and free taxol (administered in 10% DMSO) produced similar effects against the taxol-sensitive OVCA429 cell line, the C7 conjugate produced significantly enhanced benefits relative to taxol in the taxol-resistant OVCA429T cells (p=0.0002) (FIG. 3b).

We have therefore demonstrated that conjugation of octaarginine peptides to a small molecule drug (taxol) and drug surrogates (luciferin or coelenterazine H) via disulfide linkers can provide a variety of benefits, including improved administration due to enhanced aqueous solubility, altered (localized) biodistribution, lengthened pharmacokinetics and most importantly the ability to overcome multidrug resistance. In particular it was found that r8 conjugated to the C2′ position of taxol produces a highly water soluble conjugate (thereby avoiding the need for Chremophor EL), allows for sustained release of the free drug thereby minimizing peak-trough effects, enhances the cytotoxicity of the drug against a panel of cell lines, and provides significantly increased benefits in ovarian cancer mouse models relative to taxol itself. Significantly, the C7 conjugate of taxol (3a) overcomes the resistance exhibited by taxol itself.

This effect is observed for cell lines with over-expressed Pgp efflux pumps as well as for cells with complex multidrug resistance phenotypes in cell culture and in animal tumor models. Generally, if a cancer develops resistance to a drug, it is necessary to switch to a second drug to circumvent this resistance. The approach described here provides an alternative treatment strategy. Many drugs (e.g., etoposide, camptothecin, and doxorubicin) because of their hydrophobic nature are substrates for Pgp efflux pumps. Attachment of a transporter to these agents could dramatically change their physical properties and therefore mode of cell entry, thereby avoiding Pgp based resistance.

Although taxol prodrugs with aqueous solubility as well as targeted delivery have been previously described, they often require the release of the solubilizing subunit of the conjugate to allow diffusion of the drug across the non-polar plasma membrane. In contrast, the octaarginine transporter not only allows for solubilization of the conjugate in aqueous solution and the administration benefits derived there from, but it also enhances uptake and with a suitable linker allows for controlled release of free drug, factors that favor improved performance and minimize peak-trough effects. The ability to improve the administration and performance of a drug and to overcome resistance elicited by that drug through conjugation with an octaarginine transporter could improve the prognosis for the treatment of cancer with many therapeutic agents.

### Methods

Cell Lines; SKOV-3 cells were obtained from ATCC; UCI-101 and UCI-107 cell lines were obtained from Drs. P. DiSaia and A. Manetta, University of California; MCF-7 and MCF-7-Pgp were obtained from Dr. D. Piwnica-Worms, Washington University; OVCA429, OVCA433 and the taxane resistant derivatives of these cells (OVCA429T, OVCA429TxT, OVCA433T and OVCA433TxT) were obtained from Dr. B. Sikic, Stanford University. All cells were grown in DMEM with 10% FBS.

Cell Cytotoxicity Assay. The cytotoxicity of relevant compounds was determined by IC₅₀ assay. Cells were seeded overnight into 96-well plates, and then incubated with a serial dilution of the indicated compound for 20 min, before washing twice with fresh media, and incubation at 37° C. for 72 hr in drug-free media. The IC₅₀ values were determined as the concentration of compound required to inhibit the viability of the cell layer by 50% relative to untreated, and cell-free control wells (100 and 0% viability respectively) determined from semi-logarithmic dose response curves. Viability was assayed by CellTiter96 Aqueous assay (MTS) according to the manufacturer's instructions (Promega, Madison, Wis.), or as bioluminescence signal following addition of 0.3 mg/ml luciferin substrate to luciferase expressing cells. Bioluminescence was determined using an IVIS 50 system (Caliper Life Sciences, Alameda, Calif.). Each compound and dilution was tested in triplicate per experiment, with each experiment reproduced three times.

Microtubule Assembly Assay: Tubulin depolymerization assay was performed according to an adaptation of the methods described by Matthew et. al (1992) J Med Chem 35:145-151. Briefly, 2 μM tubulin protein (Cytoskeleton Inc, Denver, Colo.) was allowed to polymerize at 30° C. in the presence of 10 μM taxol, or derivative and 0.5 mM GTP in PEM buffer. Polymerization was determined as increasing turbidity, monitored by absorbance at 350 nm.

Cell Cycle Analysis: Cell cycle analysis was performed on cancer cell lines, or cells pre-treated with taxol, or its derivatives according to standard techniques. Briefly, cells were treated with taxol or a derivative at 1 mM for 15 minutes and then washed twice before incubation for 24 hr. Cells were then detached, stained with 7-AAD (7-amino-actomycin D) and analyzed by flow cytometry. Initially, doublets were removed by gating, so that only single cells were analyzed. Then the FL3 channel was analyzed, to divide the cells into G2/M, S and G1/S subsets. The percentage of cells in G2/M was recorded. Data from 3 separate experiments were combined.

Construction and testing of OVCA429 and OVCA429T cells expressing luciferase. Stable versions of the cell lines OVCA429 and OVCA429T were constructed expressing Firefly luciferase, or Firefly and Renilla luciferase enzymes. Two versions of the plasmid pcDNA3.1 were constructed, in the first, Firefly luciferase was placed under the control of the CMV promoter, and the puromycin selection gene inserted; in the second, Renilla luciferase was placed under control of the CMV promoter, and the zeocyin selection gene inserted. Firefly luciferase expression was produced by lipofectamine (Invitrogen, Carlsbad, Calif.) transfection of the appropriate plasmid according to the manufacturer's guidelines, followed by selection on puromycin. A second transfection, into the Firefly luciferase expressing cells, and using the Renilla luciferase expression plasmid and zeocyin selection produced cell lines expressing both luciferase enzymes. Stability was determined by growth in media without selection, phenotypic properties (gross morphology, taxol resistance and growth rate) were determined to ensure the gene expression did not overtly alter the characteristics of the cells. In some experiments, cells expressing Renilla luciferase were treated with coelenterazine substrate at 50 μM (a known substrate of the Pgp transporter), either alone, or in the presence of 5 μM of cyclosporine A. Light output was measured as an indicator of Pgp function, using an IVIS 50 (Xenogen Product line of Caliper Life Sciences, Alameda, Calif.). Luciferin, and Firefly luciferase bioluminescence was used to normalize the readings.

Mouse Biodistribution Studies: L2G85 transgenic mice, carrying the luciferase gene driven by the beta-actin promoter, were treated with a single intraperitoneal injection of 5 mg/kg luciferin or equimolar amounts of luciferin conjugates. Biolumninescence signal was determined at regular time points immediately after injection using an IVIS 200 system (Caliper Life Sciences, Alameda, Calif.).

Mouse Tumor Models: Tumor xenografts were created by intraperitoneal injection of female athymic CD1 nu/nu or SCID mice with 1×10⁷ cells. Versions of UCI-101, OVCA429 or OVCA429T cells were used expressing Firefly luciferase. Tumor establishment and growth was verified by increasing bioluminescence signal (as determined by in vivo bioluminescence imaging (BLI) on an IVIS100 system (Caliper Life Sciences, Alameda, Calif.), following intraperitoneal injection with 30 mg/kg luciferin and anesthesia with 2% isoflurane). Mice were then treated with three intraperitoneal injections (5 days apart) of 5 or 10 mg/kg taxol or equimolar dose equivalents of the taxol derivatives, or PBS as a control. For the purpose of these experiments TFA⁻ counteranions on both conjugates were exchanged to C. Subsequent tumor burden was followed by BLI. Once tumor burden reached 1×10⁸ photons/sec/mouse the mice were euthanized. All studies were run according to IACUC approved protocols.

Statistical Analyses; Comparisons of cell numbers in G2/M phase were made by Student's T-test. Comparisons of survival (Kaplan-Meier) curves were made by the Wilcoxon-Rank test. Statistical significance was determined as p<0.05.

General methods. Unless otherwise stated, all reagents and solvents were obtained from commercial sources and used without purification. All reagents for peptide synthesis including NMP, DIEA, DMF, HOBT, HBTU, and piperidine were purchased from Aldrich, NovaBiochem (CA), BaChem (CA), or Applied Biosystems (CA). Fmoc-protected amino acids and resins were purchased from NovaBachem or BaChem in their appropriately protected form. All automated peptide syntheses were performed on a PE Biosystems Model 433A automated peptide synthesizer using the standard FastMoc coupling strategy. Taxol was obtained from the drug repository of the National Cancer Institute (NCl) at the NIH (Bethesda, Md.). Reverse-phase high performance liquid chromatography (RP-HPLC) was performed with a Varian ProStar 210/215 HPLC using a preparative column (Alltec Alltima C18, 250×22 mm) or on an Agilent 1100 analytical HPLC with an analytical column (Vydak C18, 150×4.6 mm). The products were eluted utilizing a solvent gradient (solvent A=0.1% TFA/H₂O; solvent B=0.1% TFA/CH₃CN).

NMR spectra were measured on a Varian INOVA 500 (¹H NMR at 500 MHz; ¹³C NMR at 125 MHz) or a Varian INOVA 400 (¹H NMR at 400 MHz; ¹³C NMR at 100 MHz) magnetic resonance spectrometers. Data for ¹H NMR spectra are reported as follows: chemical shift, multiplicity (s=singlet, d=doublet, dd=doublet of doublet, t=triplet, q=quartet, and m=multiplet), coupling constant (Hz) and integration. Data for ¹H NMR spectra are reported in terms of chemical shift relative to residual solvent peak (3.31 (CD₃OD) and 7.27 (CDCl₃) ppm for ¹H NMR spectra). Matrix Assisted Laser Desorption mass spectra (MALDI) were recorded on an Applied Biosystems Voyager DE mass spectrometer. High resolution MS (HRMS) and low resolution MS were obtained at the Vincent Coates foundation mass spectrometry laboratory at Stanford University, California.

Synthesis and Characterization of Compounds Used in the Study

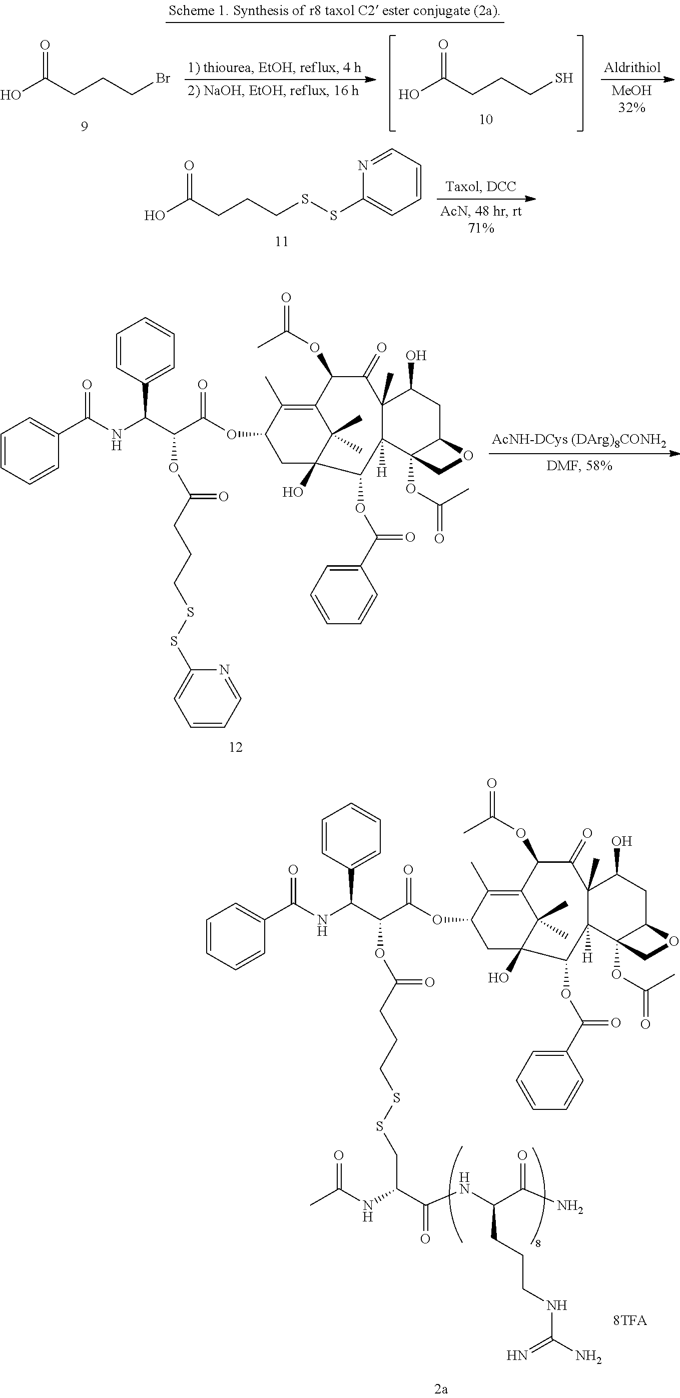

4-(Pyridin-2-yldisulfanyl)-butyric acid (11). Acid 11 was synthesized from free thiol 10¹ as previously described by us in 32% yield over 2 steps from commercially available 4-bromobutyric acid (Jones et al. (2006) J. Am. Chem. Soc. 128, 6526-6527). ¹H NMR (500 MHz, CD₃OD): δ=8.50 (d, 1H, J=4.5 Hz), 7.74 (d, 1H, J=8.0 Hz), 7.68 (t, 1H, J=8.0 Hz), 7.13 (t, 1H, J=7.0 Hz), 2.87 (t, 2H, J=7.0 Hz), 2.52 (t, 2H, J=7.0 Hz), 2.06 (m, 2H) ppm. ¹³C NMR (125 MHz, CDCl₃): δ=178.1, 159.9, 149.4, 137.3, 120.8, 119.9, 37.7, 32.3, 23.7 ppm. IR (thin film): 2924, 1715, 1575, 1417, 1217, 1120, 761 cm⁻¹. EI-MS (m/z): [M+1] calculated for [C₉H₁₂NO₂S₂] 230.0; found 230.0.

Taxol C2′ Ester 12. Procedure published by Rodrigues and coworkers were used for the coupling of acid 11 with Taxol to afford compound 12 in 71% yield (Rodrigues et al. (1995) Chem. Biol. 2, 223-227). ¹H NMR (500 MHz, CDCl₃): δ=8.46 (m, 1H), 8.16 (d, J=7.6 Hz, 2H), 7.78 (d, J=7.6 Hz, 2H), 7.69-7.35 (m, 13H), 7.11 (m, 1H) 6.99 (d, J=9.0 Hz, 1H), 6.32 (s, 1H), 6.28 (t, J=9.0 Hz, 1H), 5.99 (dd, J₁=9.1 Hz, J₂=3.2 Hz, 1H), 5.71 (d, J=7.0 Hz, 1H), 5.54 (d, J=2.2 Hz, 1H), 4.99 (d, J=9.0 Hz, 1H), 4.48 (dd, J₁=11.0 Hz, J₂=6.8 Hz, 1H)), 4.33 (d, J=8.5 Hz, 1H), 4.21 (d, J=8.5 Hz, 1H), 3.84 (d, J=7.0 Hz, 1H), 2.84-2.77 (m, 2H), 2.66-2.55 (m, 2H), 2.47 (s, 3H), 2.38 (m, 1H), 2.25 (s, 3H), 2.20 (m, 1H), 2.05 (m, 3H), 1.91 (m, 4H), 1.78 (m, 1H), 1.71 (s, 3H), 1.25 (s, 3H), 1.16 (s, 3H) ppm. ¹³C NMR (125 MHz, CDCl₃): δ=204.1, 172.1, 171.5, 170.1, 168.3, 167.5, 167.3, 159.5, 149.1, 145.0, 143.0, 138.3, 137.1, 134.0, 133.7, 133.0, 132.4, 130.6, 130.5, 129.8, 129.4, 129.0, 128.8, 128.3, 128.2, 128.1, 127.4, 126.8, 121.4, 120.8, 84.7, 81.3, 79.4, 75.9, 75.3, 74.3, 72.4, 72.1, 58.8, 53.0, 45.9, 43.4, 37.5, 35.8, 32.2, 27.1, 24.0, 23.0, 22.4, 21.1, 15.1, 9.9 ppm. IR (thin film): 3506, 2929, 1731, 1539, 1373, 1240, 1070, 709 cm⁻¹. MS (m/z): [M+2] calculated for [C₅₆H₆₂N₂O₁₅S₂] 1066.3; found (MALDI) 1066.7.

Taxol C2′ ester octaarginine conjugate 2a. This compound was coupled with Ac—NH-DCys (DArg)₈CONH₂ using a procedure previously published by us in 58% yield (Jones et al., supra). ¹H NMR (500 MHz, CD₃OD): δ=8.14 (d, J=7.5 Hz, 2H), 7.85 (d, J=7.5 Hz, 2H), 7.72 (m, 1H), 7.59 (m, 3H), 7.48 (m, 6H), 7.28 (m, 1H), 6.46 (s, 1H), 6.03 (t, J=8.5 Hz, 1H), 5.80 (d, J=7.0 Hz, 1H), 5.65 (d, J=7.0 Hz, 1H), 5.49 (m, 1H), 5.03 (d, J=9.5 Hz, 1H), 4.54 (m, 1H), 4.35-4.26 (m, 11H), 4.20 (m, 2H), 3.81 (d, J=7.0 Hz, 1H), 3.22 (m, 18H), 2.98 (m, 1H), 2.77 (t, J=7.0 Hz, 2H), 2.61 (m, 2H), 2.49 (m, 1H), 2.40 (s, 3H), 2.20 (s, 3H), 2.14-2.03 (m, 6H), 1.94-1.87 (m, 43H), 1.17 (s, 3H), 1.14 (s, 3H) ppm. MS (m/z): [M+1] calculated for [C₁₀₄H₁₆₂N₃₅O₂₅S₂] 2365.2; found (MALDI) 2365.8.

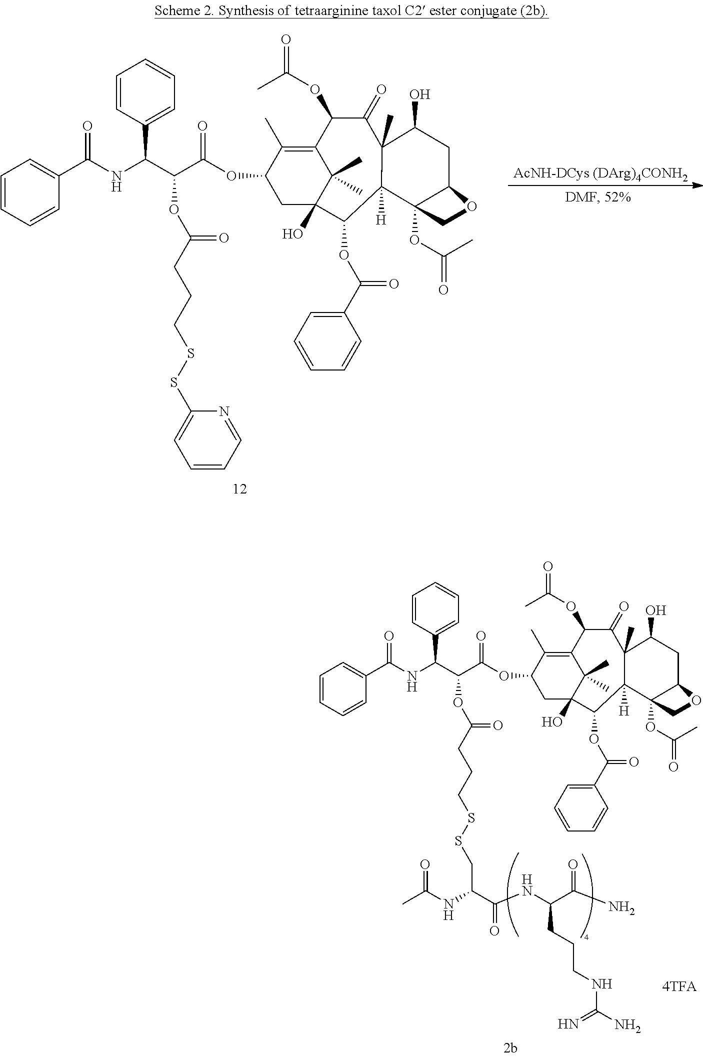

Taxol C2′ ester tetraarginine conjugate 2b. Taxol ester 12 was coupled with Ac—NH-DCys (DArg)₄CONH₂ using a procedure previously published by us in 57% yield (Jones et al., supra). ¹H NMR (500 MHz, CD₃OD): δ=8.14 (d, J=7.5 Hz, 2H), 7.86 (d, J=7.5 Hz, 2H), 7.72 (m, 1H), 7.61 (m, 3H), 7.49 (m, 6H), 7.30 (m, 1H), 6.46 (s, 1H), 6.01 (t, J=8.5 Hz, 1H), 5.82 (d, J=7.0 Hz, 1H), 5.67 (d, J=7.0 Hz, 1H), 5.49 (m, 1H), 5.04 (d, J=9.5 Hz, 1H), 4.88 (s, 1H), 4.53 (m, 1H), 4.36-4.27 (m, 6H), 4.20 (m, 2H), 3.81 (d, J=7.0 Hz, 1H), 3.22 (m, 9H), 3.14 (m, 1H), 2.98 (m, 1H), 2.77 (t, J=7.0 Hz, 2H), 2.61 (m, 2H), 2.49 (m, 1H), 2.40 (s, 3H), 2.21 (s, 3H), 2.11-2.03 (m, 6H), 1.95-1.86 (m, 27H), 1.17 (s, 3H), 1.14 (s, 3H) ppm. MS (m/z): [M+1] calculated for [C₈₀H₁₁₄N₁₉O₂₁S₂] 1740.8; found (MALDI) 1741.5

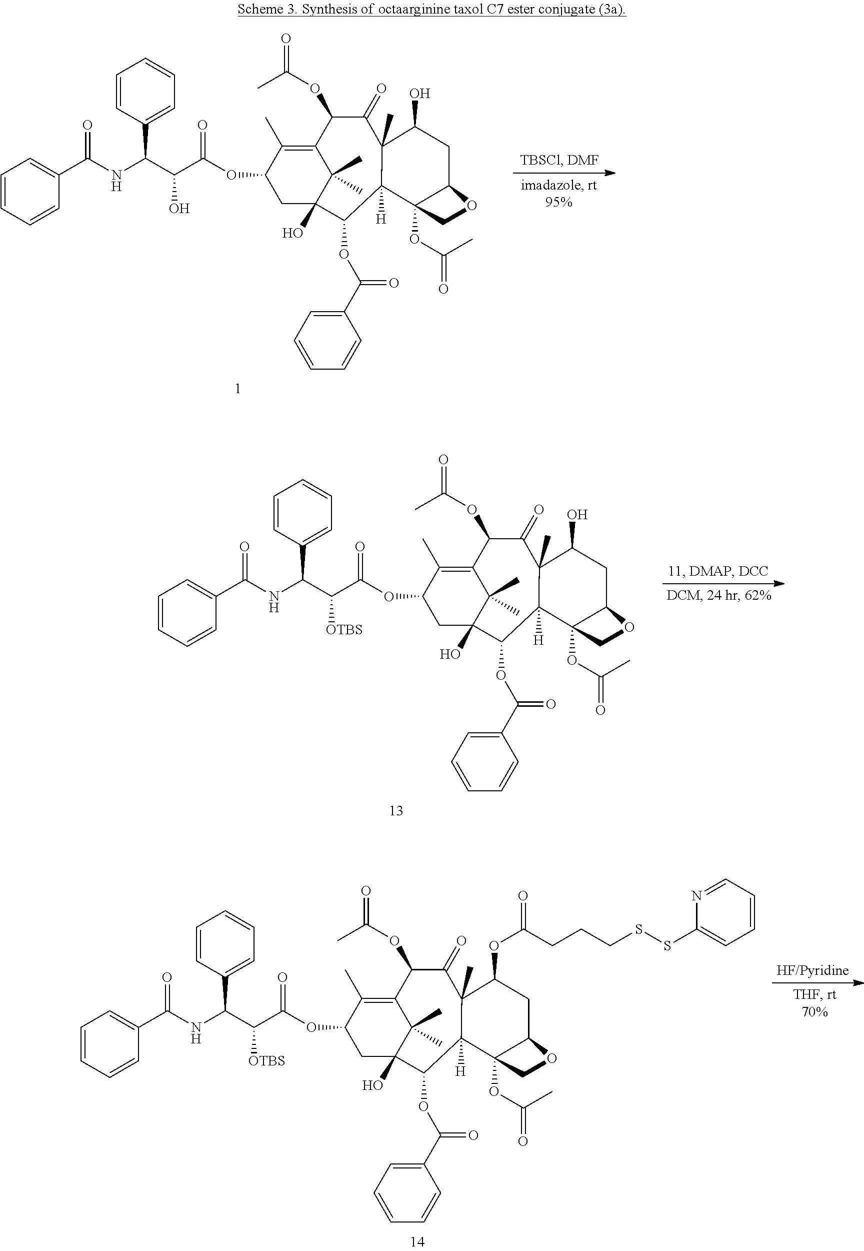

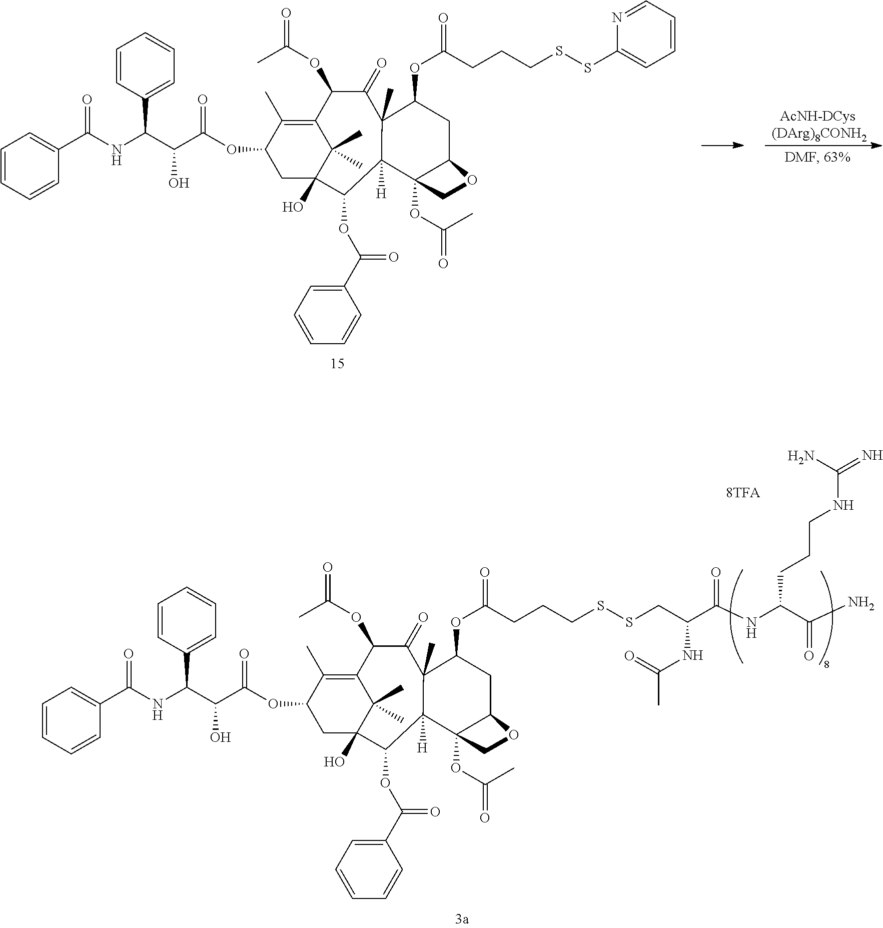

Taxol C2′ TBS Ester 13. Synthesis of C2′ TBS protected taxol 13 has been previously described by Magri et al. (1988) J. Nat. Prod. 51, 298-306 and their procedure has been followed precisely to afford TBS C2′ ester 13 in 95% yield. ¹H NMR (500 MHz, CDCl₃): δ=8.14 (d, J=10 Hz, 2H), 7.76 (d, J=10 Hz, 2H), 7.62 (m, 1H), 7.53 (m, 3H), 7.41 (m, 4H), 7.33 (m, 3H), 7.09 (d, J=9 Hz, 2H), 6.30 (t, J=8 Hz, 1H), 5.75 (d, J=8.5 Hz, 1H), 5.70 (d, J=7 Hz, 1H), 5.00 (d, J=11.5 Hz, 1H), 4.66 (d, J=2 Hz, 1H), 4.46 (dd, J₁=11 Hz, J₂=6 Hz, 1H), 4.33 (d, J=8 Hz, 1H), 4.23 (d, J=8 Hz, 1H), 3.84 (d, J=7 Hz, 1H), 2.59 (m, 4H), 2.42 (m, 1H), 2.24 (s, 3H), 2.15 (m, 1H), 1.92 (s, 3H), 1.71 (s, 3H), 1.26 (s, 3H), 1.15 (s, 3H), 0.81 (s, 9H), −0.02 (s, 3H), −0.27 (s, 3H) ppm. ¹³C NMR (125 MHz, CDCl₃): δ=204.0, 171.6 (2C), 170.4, 167.3, 167.2, 142.8, 138.5, 134.3, 134.0, 133.1, 132.1, 130.5, 129.4, 129.1, 129.0 (3C), 128.3, 127.3, 126.7, 84.7, 81.4, 79.4, 75.8, 75.5, 75.3, 72.4, 71.7, 58.8, 55.9, 45.7, 43.5, 36.0, 35.8, 27.0, 25.8, 23.3, 22.6, 21.1, 18.4, 15.2, 9.9, −5.0, −5.6 ppm. IR (thin film): 3440, 2962, 1723, 1661, 1519, 1486, 1370, 1268, 1107, 838, 710 cm⁻¹. MS (m/z): [M+1] calculated for [C₅₃H₆₆NO₁₄Si] 968.4; found (MALDI) 968.6.

Taxol C7 ester 14. Formation of ester 14 at C7 position has been done according to previously published procedure by Damen et al. (2000) Bioorg. Med. Chem. 8, 427-432 to afford the desired ester 14 in 62% yield. ¹H NMR (500 MHz, CDCl₃): δ=8.44 (m, 1H), 8.12 (d, J=10.5 Hz, 2H), 7.73 (m, 3H), 7.61 (m, 2H), 7.49 (m, 3H), 7.42-7.31 (m, 6H), 7.07 (m, 2H), 6.25 (m, 2H), 5.73-5.68 (m, 2H), 5.59 (m, 1H), 4.95 (d, J=11.5 Hz, 2H), 4.66 (s, 1H), 4.33 (d, J=10.5 Hz, 1H), 4.19 (d, J=10.5 Hz, 1H), 3.95 (d, J=8.5 Hz, 1H), 2.83 (t, J=8.0 Hz, 2H), 2.56 (s, 3H), 2.78 (m, 4H), 2.14 (s, 3H), 1.97 (s, 3H), 1.79 (m, 6H), 1.26-1.15 (m, 7H), 0.79 (s, 9H), −0.03 (s, 3H), −0.31 (s, 3H) ppm. ¹³C NMR (125 MHz, CDCl₃): δ=202.2, 172.2, 171.7, 170.1, 169.2, 167.2, 160.7, 149.9, 141.2, 138.5, 137.3, 134.3, 134.0, 132.9, 132.1, 130.5, 129.3, 129.1, 129.0, 128.2, 127.3, 126.6, 120.8, 119.8, 84.2, 81.2, 78.9, 76.7, 75.4, 75.3, 74.7, 71.6, 56.3, 55.9, 47.1, 43.6, 38.3, 35.8, 33.6, 32.7, 29.9, 26.6, 25.8, 23.8, 23.3, 21.7, 21.0, 18.4, 14.9, 11.2, 1.3, −4.9, −5.6 ppm. IR (thin film): 3441, 2951, 1724, 1665, 1370, 1241, 1069, 838 cm⁻¹. MS (m/z): [M+2] calculated for [C₆₂H₇₆N₂O₁₅S₂Si] 1180.4; found (MALDI) 1180.1.

Taxol C7 ester 15. TBS deprotection of C2′ ester to afford compound 15 in 70% yield was done using previously published procedure by Kirschberg et al. (2003) Org. Lett. 5, 3459-3462. ¹H NMR (500 MHz, CDCl₃): δ=8.44 (m, 1H), 8.11 (d, J=10.5 Hz, 2H), 7.73 (m, 3H), 7.61 (m, 2H), 7.49 (m, 3H), 7.42-7.31 (m, 6H), 7.38 (d, J=9.0 Hz, 1H), 7.08 (m, 1H) 6.21 (m, 2H), 5.81 (d, J=9.0 Hz, 1H), 5.68 (d, J=7.0 Hz, 1H), 5.55 (m, 1H), 4.95 (d, J=11.5 Hz, 2H), 4.81 (s, 1H), 4.33 (d, J=10.5 Hz, 1H), 4.19 (d, J=10.5 Hz, 1H), 3.95 (m, 2H), 2.84 (t, J=8.0 Hz, 2H), 2.54-2.43 (m, 3H), 2.38 (s, 3H), 2.34 (m, 2H), 2.17 (s, 3H), 2.05-1.95 (m, 2H), 1.82 (s, 3H), 1.80 (s, 3H), 1.78 (m, 1H), 1.21 (s, 3H), 1.17 (s, 3H) ppm. ¹³C NMR (125 MHz, CDCl₃): δ=202.2, 172.7, 172.2, 170.6, 169.2, 167.3, 167.1, 160.6, 149.8, 140.7, 138.3, 137.3, 134.1, 133.9, 133.2, 130.4, 129.3, 129.2, 129.0, 128.9, 128.5, 127.3, 120.8, 199.9, 84.1, 81.2, 78.7, 76.7, 75.5, 74.5, 73.5, 72.3, 71.7, 56.4, 55.2, 47.2, 43.5, 38.2, 35.8, 33.7, 32.7, 26.8, 23.8, 22.8, 21.1, 21.0, 14.9, 11.1 ppm. IR (thin film): 3522, 2929, 1731, 1539, 1373, 1240, 1070, 1018, 709 cm⁻¹. MS (m/z): [M+Na] calculated for [C₅₆H₆₀N₂O₁₅S₂Na] 1087.3 found (MALDI) 1087.4.

Taxol C7 ester octaarginine conjugate 3a. Taxol C7 ester 15 was coupled with Ac—NH-DCys (DArg)₈CONH₂ using a procedure previously published by us to afford the desired product in 63% yield (Jones et al., supra). ¹H NMR (500 MHz, CD₃OD): δ=8.14 (d, J=7.5 Hz, 2H), 7.88 (d, J=7.5 Hz, 2H), 7.71 (m, 1H), 7.59 (m, 3H), 7.48 (m, 6H), 7.32 (m, 1H), 6.27 (s, 1H), 6.17 (t, J=8.5 Hz, 1H), 5.67 (m, 2H), 5.61 (m, 1H), 5.04 ((d, J=9.5 Hz, 1H), 4.89 (s, 1H), 4.78 (d, J=5.5 Hz, 1H), 4.52 ((t, J=7.0 Hz, 1H), 4.35-4.21 (m, 11H), 3.93 (d, J=7.0 Hz, 1H), 3.23 (m, 18H), 3.05 (m, 1H), 2.76 (t, J=7.0 Hz, 2H), 2.55 (m, 1H), 2.41 (m, 5H), 2.28 (m, 1H), 2.18 (s, 3H), 2.08 (s, 3H), 2.03-1.65 (m, 45H), 1.18 (s, 3H), 1.14 (s, 3H) ppm. MS (m/z): [M+Na] calculated for [C₁₀₄H₁₆₁N₃₅O₂₅S₂Na] 2387.2; found (MALDI) 2387.4.

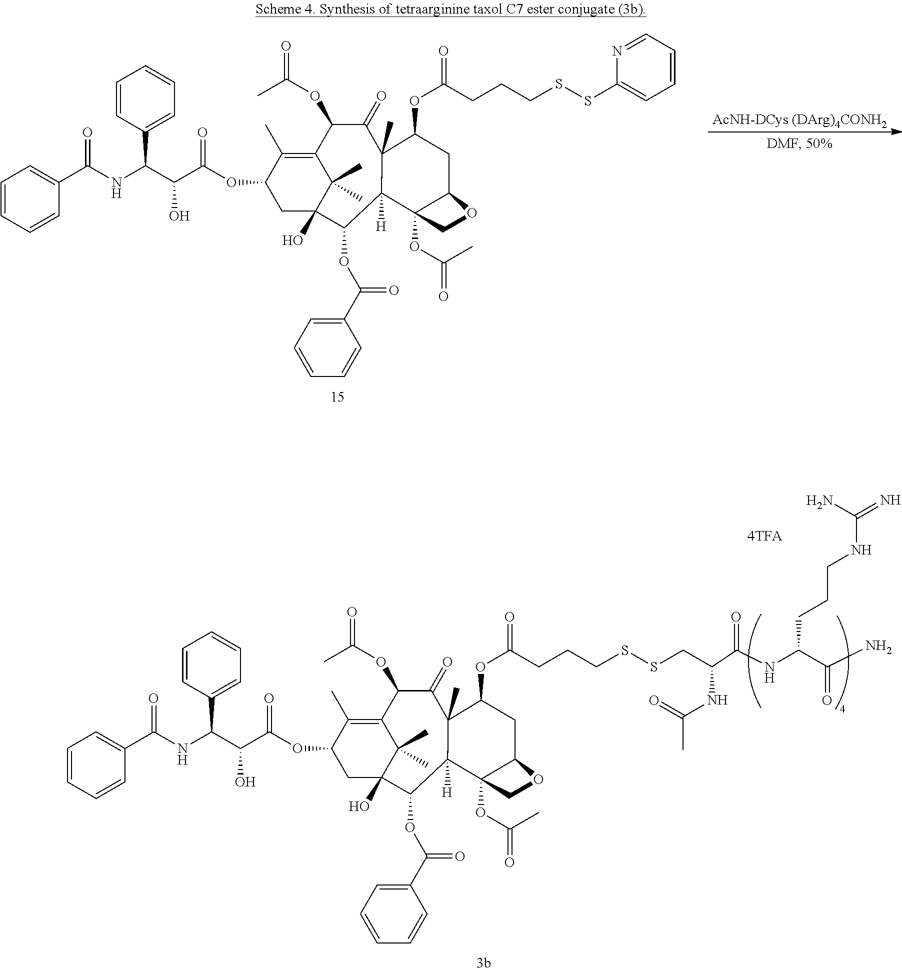

Taxol C7 ester tetraarginine conjugate 3b. Taxol C7 ester 15 was coupled with Ac—NH-DCys (DArg)₄CONH₂ using a procedure previously published by us to afford the desired product in 50% yield (Jones et al., supra). ¹H NMR (500 MHz, CD₃OD): g=8.15 (d, J=7.5 Hz, 2H), 7.88 (d, J=7.5 Hz, 2H), 7.72 (m, 1H), 7.59 (m, 3H), 7.48 (m, 6H), 7.32 (m, 1H), 6.26 (s, 1H), 6.15 (t, J=8.5 Hz, 1H), 5.64 (m, 2H), 5.61 (m, 1H), 5.05 (d, J=9.5 Hz, 1H), 4.89 (s, 1H), 4.78 (d, J=5.5 Hz, 1H), 4.50 (t, J=7.0 Hz, 1H), 4.33-4.18 (m, 6H), 3.93 (d, J=7.0 Hz, 1H), 3.30 (m, 9H), 3.03 (m, 1H), 2.77 (t, J=7.0 Hz, 2H), 2.55 (m, 1H), 2.41 (m, 5H), 2.28 (m, 1H), 2.18 (s, 3H), 2.08 (s, 3H), 2.15-1.76 (m, 29H), 1.19 (s, 3H), 1.15 (s, 3H) ppm. MS (m/z): [M+Na] calculated for [C₈₀H₁₁₃N₁₉O₂₁S₂Na] 1762.8; found (MALDI) 1763.3

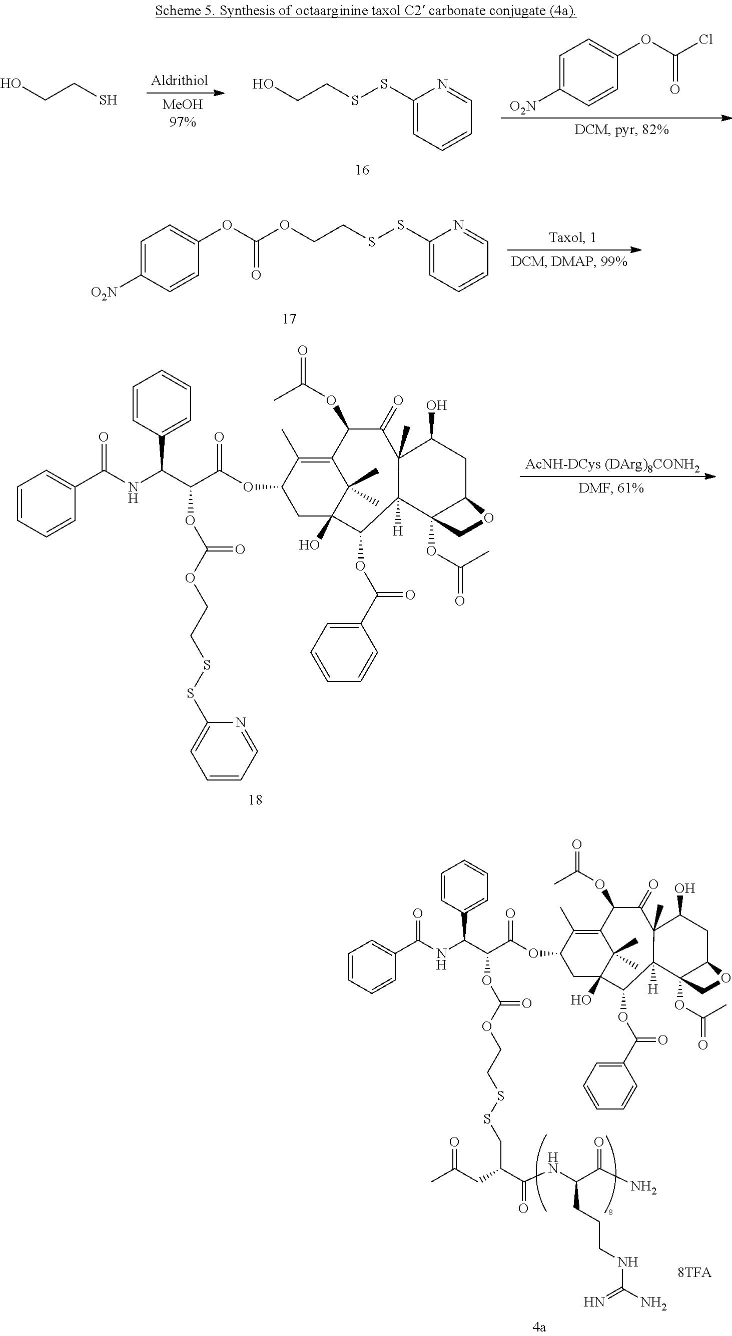

p-Nitrophenyl carbonate 17. p-Nitrophenylchloroformate was reacted with alcohol 16, according to the procedure described by Anderson et al. (1957) J. Am. Chem. Soc. 79, 6180-6183 to afford carbonate 17 in 82% yield. ¹H NMR (500 MHz, CD₃OD): δ=8.47 (m, 1H), 8.24 (dd, J₁=7.0 Hz, J₂=2.0 Hz 2H), 7.74-7.66 (m, 2H), 7.36 (dd, J₁=7.0 Hz, J₂=2.0 Hz 2H), 7.14 (m, 1H), 4.54 (t, J=6.0 Hz, 2H), 3.15 (t, J=6.0 Hz, 2H) ppm. ¹³C NMR (125 MHz, CDCl₃): δ=159.3, 155.6, 152.5, 149.8, 145.6, 137.8, 125.6, 122.1, 121.6, 120.6, 66.9, 37.0 ppm. IR (thin film): 3081, 2959, 1763, 1614, 1591, 1522, 1334, 1209, 1110, 858, 754 cm⁻¹. EI-MS (m/z): [M+1] calculated for [C₁₄H₁₃N₂O₅S₂] 353.0 found 353.0.

Taxol C2′ Carbonate 18. The synthesis of a C2′ carbonate linker was based on previously published work by de Groot et al. (2000) J. Med. Chem. 43, 3093-3102 in which p-nitrophenyl carbonate 17 was reacted with C2′ position of taxol to afford C2′ carbonate 18 in almost quantitative yield (99%). ¹H NMR (500 MHz, CD₃OD): δ=8.41 (m, 1H), 8.16 (d, J=7.6 Hz, 2H), 7.85-7.47 (m, 14H), 7.31 (m, 1H), 7.20 (m, 1H), 6.45 (s, 1H), 6.11 (t, J=8.5 Hz, 1H), 5.88 (d, J=6.0 Hz, 1H), 5.65 (d, J=7.0 Hz, 1H), 5.48 (d, J=6.5 Hz, 1H), 5.02 (d, J=9.5 Hz, 1H), 4.43 (m, 2H), 4.37 (m, 1H), 4.20 (m, 1H), 3.83 (d, 7.5 Hz, 1H), 3.12 (t, J=6.0 Hz, 2H), 2.51 (m, 1H), 2.44 (s, 3H), 2.24-2.20 (m, 4H), 1.91 (s, 3H), 1.83 (m, 2H), 1.68 (s, 3H), 1.17 (s, 3H), 1.16 (s, 3H) ppm. ¹³C NMR (100 MHz, CDCl₃): δ=203.5, 170.9, 169.5, 167.4, 166.7, 158.8, 153.5, 149.5, 142.3, 136.8, 136.3, 133.4, 133.1, 132.5, 132.1, 131.7, 130.5, 129.9, 128.8, 128.5, 128.4, 128.2, 126.8, 126.3, 120.7, 119.6, 84.1, 80.7, 78.8, 75.2, 74.7, 71.8, 67.8, 66.1, 58.2, 52.4, 45.2, 42.8, 38.3, 36.2, 35.2, 30.0, 28.6, 26.5, 23.4, 22.6, 22.4, 21.8, 20.5, 14.5, 13.7, 10.6, 9.3 ppm. IR (thin film): 3506, 2928, 1725, 1451, 1371, 1268, 1240, 1070, 709 cm⁻¹. MS (m/z): [M+1] calculated for [C₅₅H₅₉N₂O₁₆S₂] 1067.3; found (MALDI) 1067.5.

Taxol C2′ octaarginine conjugate 4a. Carbonate 18 was further coupled with Ac—NH-DCys (DArg)₈CONH₂ using a procedure previously published by us (Jones et al., supra) to afford the final conjugate in 61% yield. ¹H NMR (500 MHz, CD₃OD): δ=8.15 (d, J=7.5 Hz, 2H), 7.85 (d, J=7.5 Hz, 2H), 7.72 (m, 1H), 7.59 (m, 3H), 7.46 (m, 6H), 7.28 (m, 1H), 6.47 (s, 1H), 6.02 (t, J=8.5 Hz, 1H), 5.80 (d, J=7.0 Hz, 1H), 5.65 (d, J=7.0 Hz, 1H), 5.49 (m, 1H), 5.04 (d, J=9.5 Hz, 1H), 4.51 (m, 2H), 4.45 (m, 1H), 4.36-4.25 (m, 10H), 3.81 (d, J=7.5 Hz, 1H), 3.22 (m, 20H), 3.10 (m, 2H), 2.47 (m, 1H), 2.40 (s, 3H), 2.20 (s, 3H), 2.16 (m, 1H), 2.07 (m, 2H), 1.93-1.89 (m, 43H), 1.16 (s, 3H), 1.13 (s, 3H) ppm. MS (m/z): [M+2] calculated for [C₁₀₃H₁₆₁N₃₅O₂₆S₂] 2368.2; found (MALDI) 2368.1.

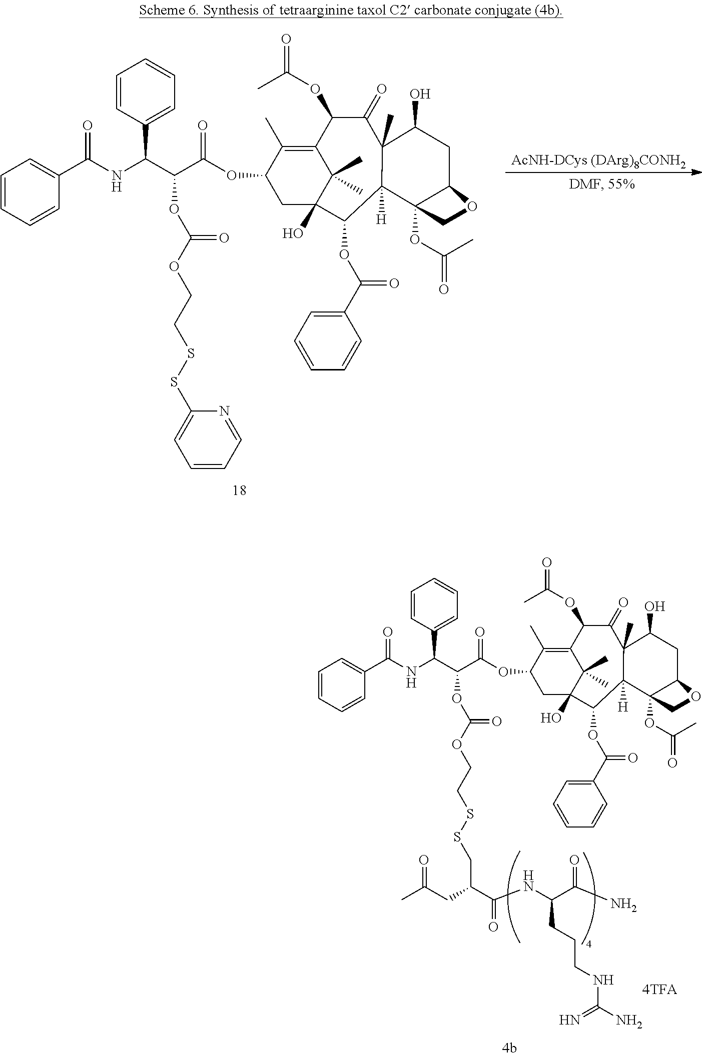

Taxol C2′ tetraarginine conjugate 4b. Taxol carbonate 18 was coupled with Ac—NH-DCys (DArg)₄CONH₂ using a procedure previously published by us in 55% yield (Jones et al., supra). ¹H NMR (500 MHz, D₂O): δ=8.05 (d, J=7.3 Hz, 2H), 7.72-7.47 (m, 6H), 7.40-7.37 (m, 6H), 7.14 (m, 1H), 6.33 (s, 1H), 5.92 (t, J=8.5 Hz, 1H), 5.60 (d, J=7.9 Hz, 1H), 5.47 (m, 2H), 5.03 (d, J=8.7 Hz, 1H), 4.42 (t, J=7.0 Hz, 1H), 4.22-4.15 (m, 7H), 3.79 (t, J=6.0 Hz, 2H), 3.65 (d, J=7.0 Hz, 1H), 3.08 (m, 10H), 2.98-2.84 (m, 2H), 2.88 (t, J=6.0 Hz, 2H), 2.45 (m, 1H), 2.29 (s, 3H), 2.13 (s, 3H), 1.87 (s, 3H), 1.81 (s, 3H), 1.81-1.51 (m, 21H), 1.08 (s, 3H), 1.03 (s, 3H).

MS (m/z): [M+1] calculated for [C₇₉H₁₁₂N₁₉O₂₂S₂] 1742.8, found (MALDI) 1742.9

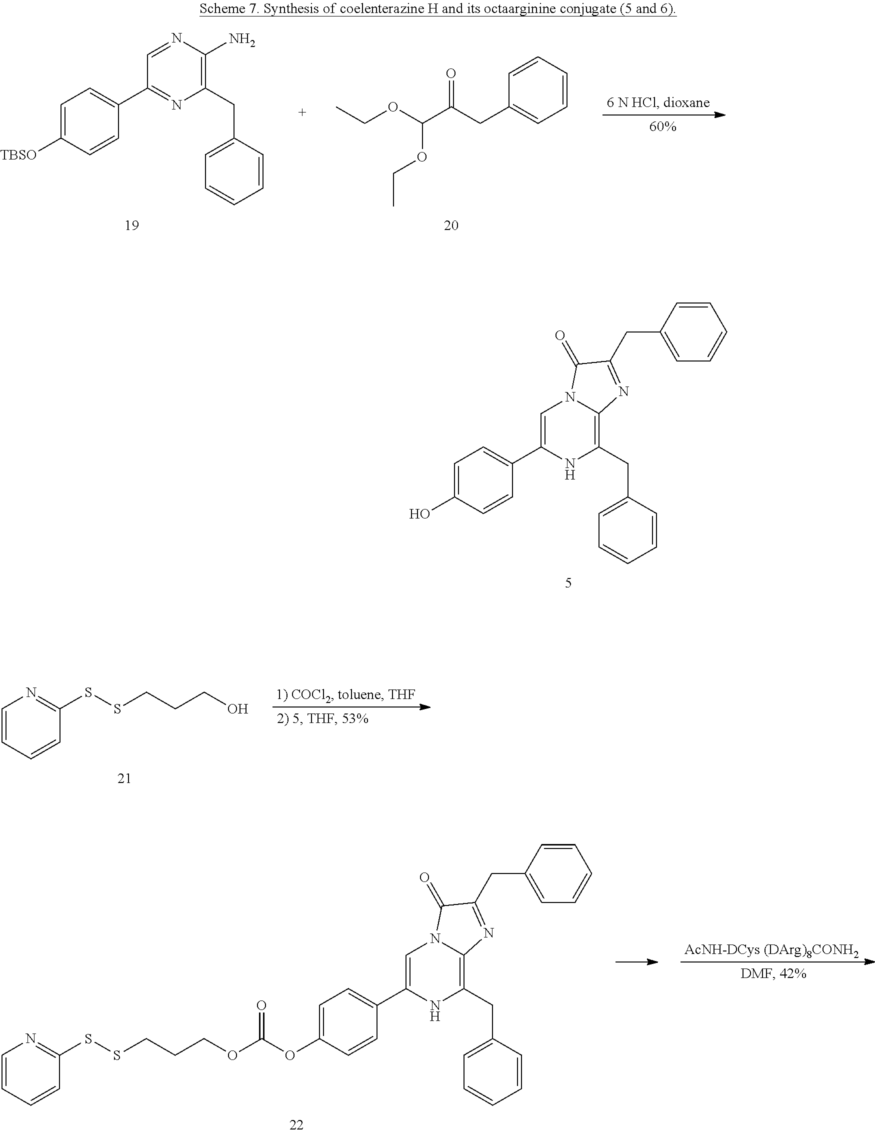

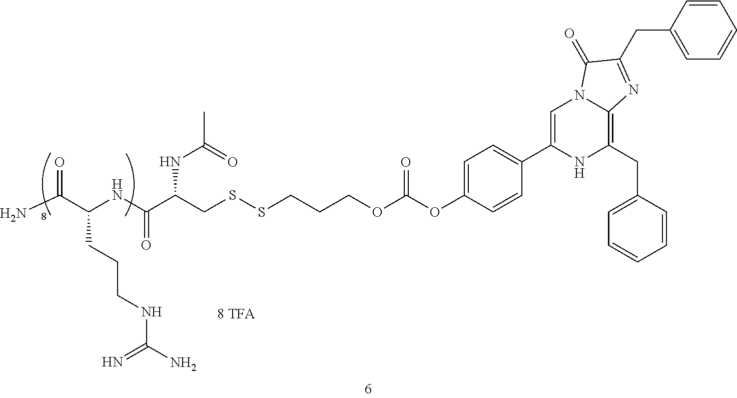

Coelenterazine H (5). Amine 19 (573 mg, 1.46 mmol) was added to an oven-dried round bottom flask equipped with a reflux condenser and a magnetic stir bar. The flask was kept under a positive pressure of nitrogen. Acetal 20 (651 mg, 2.93 mmol) was added as a solution in dioxane (22.2 mL) and then 6 N HCl (2.22 mL) was added and the solution was heated to reflux and maintained for 5 h. The reaction was then cooled to rt and quenched by addition of H₂O (100 mL). The mixture was extracted with EtOAc (3×100 mL). The combined organic phases were dried (MgSO₄), filtered, and concentrated in vacuo. Purification by flash chromatography (silica gel, 5% MeOH/CH₂Cl₂) provided 5 (356 mg, 60%) as a yellow oil. The compound was pure, providing only one spot by TLC. TLC R_{f}=0.31 (10% MeOH/CH₂Cl₂), one spot. ¹H NMR (500 MHz, CD₃OD) δ=7.49 (s, 1H), 7.34-7.38 (m, 4H), 7.30-7.32 (m, 2H) 7.20-7.25 (m, 4H), 7.10-7.17 (m, 2H), 6.80-6.83 (m, 2H), 4.35 (s, 2H), 4.14 (s, 2H) ppm. ¹³C NMR (125 MHz, CDCl₃) δ=175.7, 159.9, 139.6, 137.7, 135.9, 130.2, 129.7 (2C), 129.6 (2C), 129.5 (2C), 129.3 (2C), 129.3, 129.1 (2C), 128.0, 127.7, 127.5, 127.2, 116.7 (2C), 107.9, 42.0, 34.5 ppm. HRMS (EI m/z) Calculated for C₂₈H₂₁N₃NaO₂ (M+Na⁺): 430.1531. Found: 430.1529.

Carbonate 22. Alcohol 21 (22 mg, 0.11 mmol) was dissolved in THF (1.1 mL) in an oven-dried round bottom flask equipped with a magnetic stir bar. The solution was kept under a positive pressure of nitrogen. To this solution was added a 20% solution of phosgene in toluene (0.12 mL, 0.22 mmol). The reaction was stirred for 15 min, and then concentrated in vacuo. To the crude chloroformate was added coelenterazine H (5) (29 mg, 0.070 mmol) as a solution in THF (1.0 mL). The reaction was stirred for 12 h and then quenched by addition of H₂O (20 mL). The mixture was diluted with EtOAc (20 mL) and the separated aqueous phase was extracted with EtOAc (3×20 mL). The organic phase was then dried (MgSO₄), filtered, and concentrated in vacuo. Purification by flash chromatography (silica gel, 40% EtOAc/pentane) provided 22 (24 mg, 53%) as a yellow oil. The compound was pure, providing only one spot by TLC. TLC R_{f}=0.41 (50% EtOAc/pentane), one spot. ¹H NMR (500 MHz, CD₃OD) δ=8.42 (s, 1H), 8.37 (ddd, J=1.0, 1.7, 4.9 Hz, 1H), 7.74-7.82 (m, 4H), 7.46-7.48 (m, 2H), 7.24-7.29 (m, 6H), 7.17-7.22 (m, 3H), 6.83-6.86 (m, 2H), 4.55 (s, 2H), 4.31 (t, J=6.1 Hz, 2H), 4.18 (s, 2H), 2.87 (t, J=7.1 Hz, 2H), 2.08 (pentet, J=6.7 Hz, 2H) ppm. ¹³C NMR (125 MHz, CDCl₃) δ=159.5, 157.2, 152.6, 151.2, 149.6, 139.4, 137.7, 137.6, 137.2, 134.9, 133.0, 129.6, 128.9, 128.8, 128.5, 128.4, 128.3, 127.8, 126.5, 126.4, 121.0, 120.0, 115.9, 107.7, 68.4, 39.2, 34.5, 33.8, 27.5 ppm. HRMS (EI m/z) Calculated for C₃₅H₃₀N₄NaO₄S₂ (M+Na⁺): 657.1606. Found: 657.1605.

Coelenterazine octaarginine conjugate 6. Thiopyridine 22 (7.5 mg, 12 μmol) was added to an oven-dried vial equipped with a magnetic stir bar, and under a positive pressure of nitrogen. To this flask was added a solution of Ac—NH-DCys (DArg)₈CONH₂ (20 mg, 8.6 μmol) in DMF (0.20 mL). The reaction was stirred for 12 h, and then concentrated in vacuo. Purification by RP-HPLC (5%→>90% CH₃CN/H₂O+0.1% TFA) provided 6 (10 mg, 42%) as an amorphous pale yellow solid. The compound was pure, providing only one peak by analytical RP-HPLC. Anal. RP-HPLC: Tᵣ=8.8 min. (5%→90% CH₃CN/H₂O+0.1% TFA, 20 min), one peak. ¹H NMR (500 MHz, CD₃OD): δ=8.59-8.62 (m, 1H), 8.35 (s, 1H), 8.10-8.23 (m, 6H), 7.80-7.83 (s, 2H), 7.55 (brs, 1H), 7.46-7.48 (s, 2H), 7.18-7.31 (m, 8H), 6.85-6.88 (m, 2H), 4.55 (s, 2H), 4.51-4.53 (m, 1H), 4.21-4.33 (m, 10H), 4.17 (s, 2H), 3.15-3.22 (m, 16H), 2.86-3.00 (m, 2H), 2.78-2.80 (t, J=7.0 Hz, 2H), 2.09 (pentet, J=6.7 Hz, 2H), 2.03 (s, 3H), 1.70-1.90 (m, 32H) ppm. MS (MALDI): Calcd. for C₈₃H₁₃₄N₃₇O₁₄S₂ (M+3H): 1937.0. Found: 1937.7.

Luciferin octaarginine conjugate 7. This compound was synthesized according to previously published procedure (Jones et al, supra).

Luciferin tetralysine conjugate 8. This compound was synthesized according to previously published procedure (Wender et al. (2007) Proc. Natl. Acad. Sci. USA. 104, 10340-10345).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A method of treating a multidrug resistant cancer, the method comprising:
contacting multidrug resistant cancer cells with a chemotherapeutic drug conjugated to a, molecular transporter which conjugate has an improved therapeutic efficacy relative to the free chemotherapeutic drug.

2. The method of claim 1, wherein the molecular transporter is a peptidic transporter moiety comprising from 5 to 25 guanidino or amidino moieties

3. The method of claim 2, wherein the multidrug resistant cancer cells are contacted in vitro with the chemotherapeutic drug conjugated to a peptidic transporter moiety.

4. The method of claim 2, wherein the multidrug resistant cancer cells are contacted in vivo with the chemotherapeutic drug conjugated to a peptidic transporter moiety.

5. The method of claim 2, wherein the multidrug resistant cancer cells are tested for expression of an efflux proton pump or exclusion of an efflux proton pump substrate prior to the contacting.

6. The method of claim 2, wherein the multidrug resistant cancer cells comprise cancer stem cells.

7. The method of claim 5, wherein the efflux proton pump is p-glycoprotein.

8. The method of claim 7, wherein at least 10% of the cancer cells to be treated are multidrug resistant.

9. The method of claim 7, wherein the chemotherapeutic drug is conjugated to a peptidic transporter moiety by a releasable linker.

10. The method of claim 7, wherein the chemotherapeutic drug conjugated to a peptidic transporter moiety has the structure of formula I 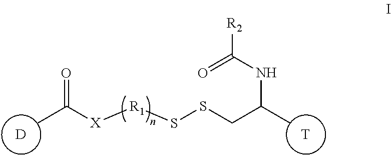
where X is CH₂; C(CH₃)₂; O; NH; or S;
R₁ is CH₂; C(CH₃)₂; C(C₂H₅)₂, or a combination thereof;
R₂ is CH₃, any alkyl chain, e.g. a C₁-C₆ lower alkyl, amino acid or peptide;
n=0-5;
D is a chemotherapeutic drug; and
T is a molecular transporter moiety.

11. The method of claim 7, wherein the chemotherapeutic drug is a p-glycoprotein substrate.

12. The method of claim 11, wherein the chemotherapeutic drug is a taxane.

13. The method of claim 12, wherein the linker has the structure of formula III 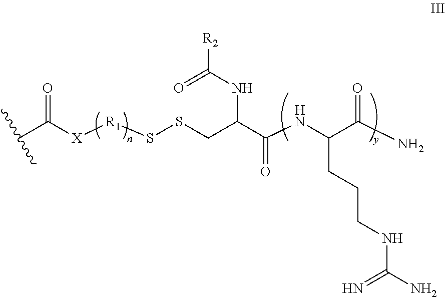
where X is CH₂; C(CH₃)₂; O; NH; or S;
R₁ is CH₂; C(CH₃)₂; C(C₂H₅)₂ or a combination thereof;
R₂ is CH₃, any alkyl chain, e.g. a C₁-C₆ lower alkyl, amino acid or peptide;
n is from 0 to 5; and
y is from 5-12.

14. The method of claim 13, wherein y is 8.

15. The method of claim 13, wherein n is 3.

16. The method of claim 15, wherein at least one arginine is a D-arginine.

17. A chemotherapeutic drug conjugate having the structure of formula I 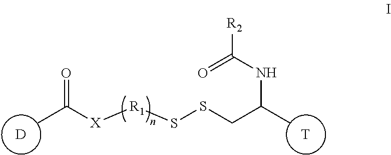
where X is CH₂; C(CH₃)₂; O; NH; or S;
R₁ is CH₂; C(CH₃)₂; C(C₂H₅)₂ or a combination thereof;
R₂ is CH₃, any alkyl chain, e.g. a C₁-C₆ lower alkyl, amino acid or peptide;
n=0-5;
D is a chemotherapeutic drug; and
T is a molecular transporter moiety.

18. The chemotherapeutic drug conjugate of claim 17, wherein the chemotherapeutic drug is a p-glycoprotein substrate.

19. The chemotherapeutic drug conjugate of claim 17, wherein the chemotherapeutic drug is a taxane.

20. The chemotherapeutic drug conjugate of claim 19, wherein the linker is conjugated to the taxane at C7, C10 or C2′ position.

21. The chemotherapeutic drug conjugate of claim 20, wherein the taxane is paclitaxel.

22. The chemotherapeutic drug conjugate of claim 21, wherein the linker is conjugated at the C2′ position.

23. The chemotherapeutic drug conjugate of claim 18, wherein the linker has the structure of formula III 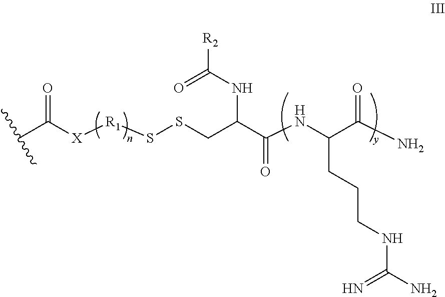
where X is CH₂; C(CH₃)₂; O; NH; or S;
R₁ is CH₂; C(CH₃)₂; C(C₂H₅)₂ or a combination thereof;
R₂ is CH₃, any alkyl chain, e.g. a C₁-C₆ lower alkyl, amino acid or peptide;
n is from 0 to 5; and
y is from 5-12.

24. The chemotherapeutic drug conjugate of claim 23, wherein y is 8.

25. The chemotherapeutic drug conjugate of claim 24, wherein n is 3.

26. The chemotherapeutic drug conjugate of claim 23, wherein at least one arginine is a D-arginine.


SEQUENCE LISTING (3 sequences)

SEQ ID NO: 1 - protein, 9 aa, Artificial Sequence
  synthetic peptide
RKKRRQRRR

SEQ ID NO: 2 - DNA, 4718 bp, homo sapiens
aaacacttgtattaccattttaaaggctatcattactctttacctgtgaagagtagaacatgaagaaatctactttattcagatattctccagattcctaaagattagagatcatttctcattctcctaggagtactcacttcaggaagcaaccagataaaagagaggtgcaacggaagccagaacattcctcctggaaattcaacctgtttcgcagtttctcgaggaatcagcattcagtcaatccgggccgggagcagtcatctgtggtgaggctgattggctgggcaggaacagcgccggggcgtgggctgagcacagccgcttcgctctctttgccacaggaagcctgagctcattcgagtagcggctcttccaagctcaaagaagcagaggccgctgttcgtttcctttaggtctttccactaaagtcggagtatcttcttccaaaatttcacgtcttggtggccgttccaaggagcgcgaggtcggaatggatcttgaaggggaccgcaatggaggagcaaagaagaagaacttttttaaactgaacaataaaagtgaaaaagataagaaggaaaagaaaccaactgtcagtgtattttcaatgtttcgctattcaaattggcttgacaagttgtatatggtggtgggaactttggctgccatcatccatggggctggacttcctctcatgatgctggtgtttggagaaatgacagatatctttgcaaatgcaggaaatttagaagatctgatgtcaaacatcactaatagaagtgatatcaatgatacagggttcttcatgaatctggaggaagacatgaccaggtatgcctattattacagtggaattggtgctggggtgctggttgctgcttacattcaggtttcattttggtgcctggcagctggaagacaaatacacaaaattagaaaacagttttttcatgctataatgcgacaggagataggctggtttgatgtgcacgatgttggggagcttaacacccgacttacagatgatgtctccaagattaatgaaggaattggtgacaaaattggaatgttctttcagtcaatggcaacatttttcactgggtttatagtaggatttacacgtggttggaagctaacccttgtgattttggccatcagtcctgttcttggactgtcagctgctgtctgggcaaagatactatcttcatttactgataaagaactcttagcgtatgcaaaagctggagcagtagctgaagaggtcttggcagcaattagaactgtgattgcatttggaggacaaaagaaagaacttgaaaggtacaacaaaaatttagaagaagctaaaagaattgggataaagaaagctattacagccaatatttctataggtgctgctttcctgctgatctatgcatcttatgctctggccttctggtatgggaccaccttggtcctctcaggggaatattctattggacaagtactcactgtattcttttctgtattaattggggcttttagtgttggacaggcatctccaagcattgaagcatttgcaaatgcaagaggagcagcttatgaaatcttcaagataattgataataagccaagtattgacagctattcgaagagtgggcacaaaccagataatattaagggaaatttggaattcagaaatgttcacttcagttacccatctcgaaaagaagttaagatcttgaagggtctgaacctgaaggtgcagagtgggcagacggtggccctggttggaaacagtggctgtgggaagagcacaacagtccagctgatgcagaggctctatgaccccacagaggggatggtcagtgttgatggacaggatattaggaccataaatgtaaggtttctacgggaaatcattggtgtggtgagtcaggaacctgtattgtttgccaccacgatagctgaaaacattcgctatggccgtgaaaatgtcaccatggatgagattgagaaagctgtcaaggaagccaatgcctatgactttatcatgaaactgcctcataaatttgacaccctggttggagagagaggggcccagttgagtggtgggcagaagcagaggatcgccattgcacgtgccctggttcgcaaccccaagatcctcctgctggatgaggccacgtcagccttggacacagaaagcgaagcagtggttcaggtggctctggataaggccagaaaaggtcggaccaccattgtgatagctcatcgtttgtctacagttcgtaatgctgacgtcatcgctggtttcgatgatggagtcattgtggagaaaggaaatcatgatgaactcatgaaagagaaaggcatttacttcaaacttgtcacaatgcagacagcaggaaatgaagttgaattagaaaatgcagctgatgaatccaaaagtgaaattgatgccttggaaatgtcttcaaatgattcaagatccagtctaataagaaaaagatcaactcgtaggagtgtccgtggatcacaagcccaagacagaaagcttagtaccaaagaggctctggatgaaagtatacctccagtttccttttggaggattatgaagctaaatttaactgaatggccttattttgttgttggtgtattttgtgccattataaatggaggcctgcaaccagcatttgcaataatattttcaaagattataggggtttttacaagaattgatgatcctgaaacaaaacgacagaatagtaacttgttttcactattgtttctagcccttggaattatttcttttattacatttttccttcagggtttcacatttggcaaagctggagagatcctcaccaagcggctccgatacatggttttccgatccatgctcagacaggatgtgagttggtttgatgaccctaaaaacaccactggagcattgactaccaggctcgccaatgatgctgctcaagttaaaggggctataggttccaggcttgctgtaattacccagaatatagcaaatcttgggacaggaataattatatccttcatctatggttggcaactaacactgttactcttagcaattgtacccatcattgcaatagcaggagttgttgaaatgaaaatgttgtctggacaagcactgaaagataagaaagaactagaaggttctgggaagatcgctactgaagcaatagaaaacttccgaaccgttgtttctttgactcaggagcagaagtttgaacatatgtatgctcagagtttgcaggtaccatacagaaactctttgaggaaagcacacatctttggaattacattttccttcacccaggcaatgatgtatttttcctatgctggatgtttccggtttggagcctacttggtggcacataaactcatgagctttgaggatgttctgttagtattttcagctgttgtctttggtgccatggccgtggggcaagtcagttcatttgctcctgactatgccaaagccaaaatatcagcagcccacatcatcatgatcattgaaaaaacccctttgattgacagctacagcacggaaggcctaatgccgaacacattggaaggaaatgtcacatttggtgaagttgtattcaactatcccacccgaccggacatcccagtgcttcagggactgagcctggaggtgaagaagggccagacgctggctctggtgggcagcagtggctgtgggaagagcacagtggtccagctcctggagcggttctacgaccccttggcagggaaagtgctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagattgtgagggcagcaaaggaggccaacatacatgccttcatcgagtcactgcctaataaatatagcactaaagtaggagacaaaggaactcagctctctggtggccagaaacaacgcattgccatagctcgtgcccttgttagacagcctcatattttgcttttggatgaagccacgtcagctctggatacagaaagtgaaaaggttgtccaagaagccctggacaaagccagagaaggccgcacctgcattgtgattgctcaccgcctgtccaccatccagaatgcagacttaatagtggtgtttcagaatggcagagtcaaggagcatggcacgcatcagcagctgctggcacagaaaggcatctatttttcaatggtcagtgtccaggctggaacaaagcgccagtgaactctgactgtatgagatgttaaatactttttaatatttgtttagatatgacatttattcaaagttaaaagcaaacacttacagaattatgaagaggtatctgtttaacatttcctcagtcaagttcagagtcttcagagacttcgtaattaaaggaacagagtgagagacatcatcaagtggagagaaatcatagtttaaactgcattataaattttataacagaattaaagtagattttaaaagataaaatgtgtaattttgtttatattttcccatttggactgtaactgactgccttgctaaaagattatagaagtagcaaaaagtattgaaatgtttgcataaagtgtctataataaaactaaactttcatgtgaaa

SEQ ID NO: 3 - protein, 1280 aa, homo sapiens
MDLEGDRNGGAKKKNFFKLNNKSEKDKKEKKPTVSVFSMFRYSNWLDKLYMVVGTLAAIIHGAGLPLMMLVFGEMTDIFANAGNLEDLMSNITNRSDINDTGFFMNLEEDMTRYAYYYSGIGAGVLVAAYIQVSFWCLAAGRQIHKIRKQFFHAIMRQEIGWFDVHDVGELNTRLTDDVSKINEGIGDKIGMFFQSMATFFTGFIVGFTRGWKLTLVILAISPVLGLSAAVWAKILSSFTDKELLAYAKAGAVAEEVLAAIRTVIAFGGQKKELERYNKNLEEAKRIGIKKAITANISIGAAFLLIYASYALAFWYGTTLVLSGEYSIGQVLTVFFSVLIGAFSVGQASPSIEAFANARGAAYEIFKIIDNKPSIDSYSKSGHKPDNIKGNLEFRNVHFSYPSRKEVKILKGLNLKVQSGQTVALVGNSGCGKSTTVQLMQRLYDPTEGMVSVDGQDIRTINVRFLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQVALDKARKGRTTIVIAHRLSTVRNADVIAGFDDGVIVEKGNHDELMKEKGIYFKLVTMQTAGNEVELENAADESKSEIDALEMSSNDSRSSLIRKRSTRRSVRGSQAQDRKLSTKEALDESIPPVSFWRIMKLNLTEWPYFVVGVFCAIINGGLQPAFAIIFSKIIGVFTRIDDPETKRQNSNLFSLLFLALGIISFITFFLQGFTFGKAGEILTKRLRYMVFRSMLRQDVSWFDDPKNTTGALTTRLANDAAQVKGAIGSRLAVITQNIANLGTGIIISFIYGWQLTLLLLAIVPIIAIAGVVEMKMLSGQALKDKKELEGSGKIATEAIENFRTVVSLTQEQKFEHMYAQSLQVPYRNSLRKAHIFGITFSFTQAMMYFSYAGCFRFGAYLVAHKLMSFEDVLLVFSAVVFGAMAVGQVSSFAPDYAKAKISAAHIIMIIEKTPLIDSYSTEGLMPNTLEGNVTFGEVVFNYPTRPDIPVLQGLSLEVKKGQTLALVGSSGCGKSTVVQLLERFYDPLAGKVLLDGKEIKRLNVQWLRAHLGIVSQEPILFDCSIAENIAYGDNSRVVSQEEIVRAAKEANIHAFIESLPNKYSTKVGDKGTQLSGGQKQRIAIARALVRQPHILLLDEATSALDTESEKVVQEALDKAREGRTCIVIAHRLSTIQNADLIVVFQNGRVKEHGTHQQLLAQKGIYFSMVSVQAGTKRQ